(19) United States
(12) Patent Application Publication
(10) Pub. No.: US 2023/0075095 A1
(43) Pub. Date: Mar. 9, 2023

(54) POLYPEPTIDES AND THEIR USE

(71) Applicant: University of Washington, Seattle, WA (US)

(72) Inventors: Neil P. KING, Seattle, WA (US); Carl Walkey, Seattle, WA (US); Jing Yang Wang, Seattle, WA (US); Brooke Fiala, Seattle, WA (US); David Veesler, Seattle, WA (US); Alexandra C. Walls, Seattle, WA (US); Una Nattermann, Seattle, WA (US)

(21) Appl. No.: 17/760,180

(22) PCT Filed: Feb. 12, 2021
(86) PCT No.: PCT/US2021/017856

§ 371 Date: Aug. 4, 2022

Related U.S. Application Data
(60) Provisional application No. 62/977,036, filed on Feb. 14, 2020.

(51) Int. Cl. C07K 14/005; C12N 15/63; G16B 20/50

(52) U.S. Cl. CPC C07K 14/005; C12N 15/63; G16B 20/50; C07K 2319/35; C07K 2319/40; C07K 2319/03

(57) ABSTRACT

Polypeptides are disclosed herein having significantly improved secretion ability from eukaryotic cells, together with fusion proteins, nanoparticles, and uses thereof, and methods for designing such polypeptides.

### CROSS REFERENCE

This application claims priority to U.S. Provisional Application Ser. No. 62/977,036 filed Feb. 14, 2020, incorporated by reference herein in its entirety.

### FEDERAL FUNDING STATEMENT

This invention was made with government support under Grant No. HDTRA1-18-1-0001, awarded by the Defense Threat Reduction Agency and Grant Nos. HHSN272201700059C and R01 GM120553, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Feb. 11, 2021, having the file name “20-1008-PCT2_SeqList_ST25.txt” and is 161 kb in size.

### BACKGROUND

Many proteins, including but not limited to viral glycoprotein antigens, must be expressed as secreted proteins in eukaryotic cells. This requirement can derive from many different causes, including but not limited to a requirement for post-translational modifications including but not limited to N-linked glycosylation, disulfide bond formation, etc. However, the yield of secreted protein from eukaryotic cells varies widely for reasons that are not fully understood by those of skill in the art, and some proteins altogether fail to secrete at appreciable levels.

### SUMMARY

In one aspect, the disclosure provides polypeptides comprising or consisting of:

(a) an amino acid sequence at least 75%, 80%, 85%, 90%, 91%, 92%, 93%, 94%, 95%, 96%, 97%, 98%, or 99% identical to the amino acid sequence of SEQ ID NO:1 (I3-01 wild type), wherein 1, 2, 3, 4, 5, 6, 7, 8, 9, or all 10 of the following mutations relative to SEQ ID NO:1 are present in the polypeptide: F32Y, H37D/E/K/N/Q/R, F43Q, F168D/E/K/N/Q/R/S/T/Y, K169D/E/N/Q, L173D/E/N/Q/S, A174S, S179D/E, K183D/E, and/or T185D/E/K/N/Q/S;

(b) an amino acid sequence at least 75%, 80%, 85%, 90%, 91%, 92%, 93%, 94%, 95%, 96%, 97%, 98%, or 99% identical to the amino acid sequence of SEQ ID NO:2 (O43-38 tetramer wild type), wherein 1, 2, 3, 4, 5, 6, 7, 8, or all 9 of the following mutations relative to SEQ ID NO:2 are present in the polypeptide: M138D/E/K/N/Q/R/S/T, L139D/N/S, A141S, V142R/T, A143S, N146D/E/K/R, R147N, H172D/E/K/N/Q, and/or E173D/K.

(c) an amino acid sequence at least 75%, 80%, 85%, 90%, 91%, 92%, 93%, 94%, 95%, 96%, 97%, 98%, or 99% identical to the amino acid sequence of SEQ ID NO:3 (O43-38 trimer wild type), wherein 1, 2, 3, 4, 5, 6, 7, 8, 9, 10, 11, 12, 13, 14, 15, 16, 17, 18, 19, 20, or all 21 of the following mutations relative to SEQ ID NO:3 are present in the polypeptide: R17D/E/K/N/Q/S/T, N19D/E, S20D/E/K/N, V21D/T, V22D/E/Q/S/T, L23D/E/K/N/Q/R/S, A26S, K27N/Q, A30S V31N/S/T, F32R/Y, L33D/E/K/N/Q/R/S/T, H37D/E/K/N/R, F43Q, W167D/E/K/N/Q/R/S/T/Y, F168D/E/K/N/Q/R/S/T/Y, K169D/E/N, L173D/E/N/Q/R/S, A174S, S179D/E/K/N/Q/R, and/or K183D/E/N/Q;

(d) an amino acid sequence at least 75%, 80%, 85%, 90%, 91%, 92%, 93%, 94%, 95%, 96%, 97%, 98%, or 99% identical to the amino acid sequence of SEQ ID NO:4 (I53_dn5A wild type), wherein 1, 2, 3, 4, 5, 6, 7, 8, 9, or all 10 of the following mutations relative to SEQ ID NO:4 are present in the polypeptide: R17T, W18D/E/K/N/Q/R/S/T/Y, N19E, E21D, L28D/E/K/N/Q/R/S/T/Y, L31D/E/K/N/Q/S/T, K32D/E/N/Q, T118D/E/N/Q/S, L120D/E/K/N/Q/R/S/T, and/or T121D/E/K/N/S; or

(e) an amino acid sequence at least 75%, 80%, 85%, 90%, 91%, 92%, 93%, 94%, 95%, 96%, 97%, 98%, or 99% identical to the amino acid sequence of SEQ ID NO:5 or 6 (hMPV wild type), wherein 1, 2, 3, 4, or all 5, of the following mutations relative to SEQ ID NO:5 or 6 are present in the polypeptide: A107D, V112R, T114E, V118R, and/or G265DG264D;

wherein residues in parentheses are optional and may be present or may be absent in whole or in part.

In another aspect, the disclosure provides fusion proteins comprising:

(a) the polypeptide according to any embodiment of the first aspect of the disclosure; and

(b) a second functional polypeptide.

In a further aspect, the disclosure provides nanoparticles comprising a plurality of the polypeptides or fusion proteins of any embodiment of the first aspect and second aspect of the disclosure, and compositions comprising a plurality of such nanoparticles. In further aspects, the disclosure provides nucleic acids encoding the polypeptides or fusion proteins, expression vectors comprising the nucleic acids operatively linked to a suitable control sequence, host cells comprising the polypeptides, fusion proteins, nanoparticles, compositions, nucleic acids, and/or expression vectors, and pharmaceutical compositions thereof.

In a further aspect, the disclosure provides computer-implemented methods for designing a secreted peptide, such as the polypeptides of the disclosure.

### DESCRIPTION OF THE FIGURES

FIG. 1(a-c). Western blots of cell supernatants of cultures transfected with degreaser variants. (a) Wild-type I3-01 (Hsia et al., Nature 2016) secretes poorly from HEK293F cells, whereas the H35D and L171Q single mutations significantly improve the yield of secreted protein. (b) The original I53_dn5 pentamer protein secretes poorly from HEK29F cells, but a single W16E mutation (I53_dn5A W16E) significantly boosts secretion. (c) The wild-type protein from which the O43-38 tetramer was derived, with a mutation made to remove an N-linked glycosylation motif, (“FucA N29S”) secretes strongly, but the O43-38 tetramer does not. The A141E degreaser mutation to the O43-38 tetramer in addition significantly boosts secretion. Protein standard is the BIO-RAD Precision Plus WesternC™ Standard; primary antibodies either anti-myc or anti-HIS/HRP conjugate.

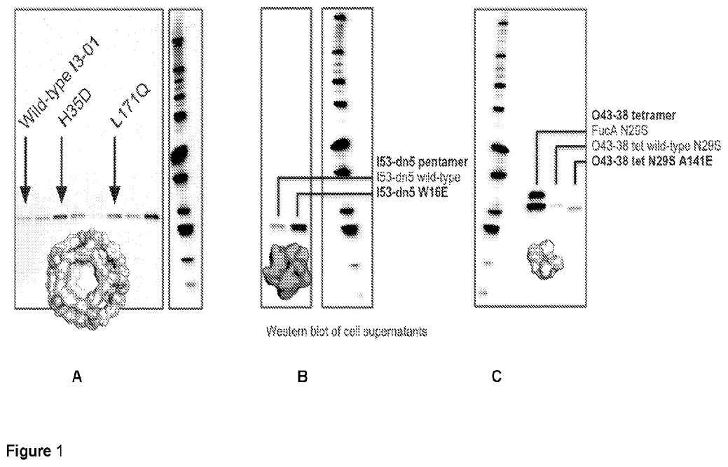

FIG. 2(a-b). Transmission electron micrographs of degreased I3-01 constructs. The H35D variant (a) as well as the H35D/L171Q/S177E/V180N quadruple mutant (b) both assemble to the expected icosahedral nanoparticle structure, confirming that the degreasing mutations do not deleteriously affect the three-dimensional structures of the proteins. Images taken at 22,000× magnification.

FIG. 3. Comparison of secreted yield by ELISA. A series of hMPV F variants fused to I53-50A with or without degreaser mutations in the hMPV F antigen was evaluated for secretion from mammalian cells; the I53-50A domain in all constructs was identical. hMPV_F-50A_14, which contains four degreaser mutations (compared to two degreaser mutations each in hMPV_F-50A_13 and hMPV_F-50A_15) significantly boosted secretion relative to constructs lacking the degreaser mutations.

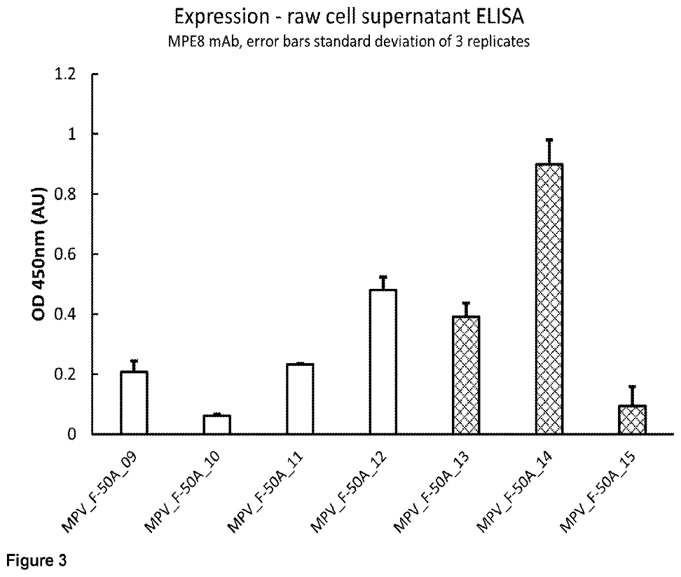

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in

Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising or consisting of:

(a) an amino acid sequence at least 75%, 80%, 85%, 90%, 91%, 92%, 93%, 94%, 95%, 96%, 97%, 98%, or 99% identical to the amino acid sequence of SEQ ID NO:1 (I3-01 wild type), wherein 1, 2, 3, 4, 5, 6, 7, 8, 9, or all 10 of the following mutations relative to SEQ ID NO:1 are present in the polypeptide: F32Y, H37D/E/K/N/Q/R, F43Q, F168D/E/K/N/Q/R/S/T/Y, K169D/E/N/Q, L173D/E/N/Q/S, A174S, S179D/E, K183D/E, and/or T185D/E/K/N/Q/S;

(b) an amino acid sequence at least 75%, 80%, 85%, 90%, 91%, 92%, 93%, 94%, 95%, 96%, 97%, 98%, or 99% identical to the amino acid sequence of SEQ ID NO:2 (O43-38 tetramer wild type), wherein 1, 2, 3, 4, 5, 6, 7, 8, or all 9 of the following mutations relative to SEQ ID NO:2 are present in the polypeptide: M138D/E/K/N/Q/R/S/T, L139D/N/S, A141S, V142R/T, A143S, N146D/E/K/R, R147N, H172D/E/K/N/Q, and/or E173D/K.

(c) an amino acid sequence at least 75%, 80%, 85%, 90%, 91%, 92%, 93%, 94%, 95%, 96%, 97%, 98%, or 99% identical to the amino acid sequence of SEQ ID NO:3 (O43-38 trimer wild type), wherein 1, 2, 3, 4, 5, 6, 7, 8, 9, 10, 11, 12, 13, 14, 15, 16, 17, 18, 19, 20, or all 21 of the following mutations relative to SEQ ID NO:3 are present in the polypeptide: R17D/E/K/N/Q/S/T, N19D/E, S20D/E/K/N, V21D/T, V22D/E/Q/S/T, L23D/E/K/N/Q/R/S, A26S, K27N/Q, A30S V31N/S/T, F32R/Y, L33D/E/K/N/Q/R/S/T, H37D/E/K/N/R, F43Q, W167D/E/K/N/Q/R/S/T/Y, F168D/E/K/N/Q/R/S/T/Y, K169D/E/N, L173D/E/N/Q/R/S, A174S, S179D/E/K/N/Q/R, and/or K183D/E/N/Q;

(d) an amino acid sequence at least 75%, 80%, 85%, 90%, 91%, 92%, 93%, 94%, 95%, 96%, 97%, 98%, or 99% identical to the amino acid sequence of SEQ ID NO:4 (I53_dn5A wild type), wherein 1, 2, 3, 4, 5, 6, 7, 8, 9, or all 10 of the following mutations relative to SEQ ID NO:4 are present in the polypeptide: R17T, W18D/E/K/N/Q/R/S/T/Y, N19E, E21D, L28D/E/K/N/Q/R/S/T/Y, L31D/E/K/N/Q/S/T, K32D/E/N/Q, T118D/E/N/Q/S, L120D/E/K/N/Q/R/S/T, and/or T121D/E/K/N/S; or

(e) an amino acid sequence at least 75%, 80%, 85%, 90%, 91%, 92%, 93%, 94%, 95%, 96%, 97%, 98%, or 99% identical to the amino acid sequence of SEQ ID NO:5 or 6 (hMPV wild type), wherein 1, 2, 3, 4, or all 5, of the following mutations relative to SEQ ID NO:5 or 6 are present in the polypeptide: A107D, V112R, T114E, V118R, and/or G264D;

wherein residues in parentheses are optional and may be present or may be absent in whole or in part.

The gatekeeper of the first step in the secretory pathway, cotranslational translocation across the ER membrane, is the Sec translocon, which acts as a fate-determining channel for nascent polypeptides. As detailed in the examples below, the inventors provide a method for improving the secretion of proteins from eukaryotic cells, and corresponded novel proteins that have improved secretion capability in eukaryotic cells, and fusion proteins and nanoparticles comprising the polypeptides, all of which can be used, for example as scaffolds for multivalent antigen presentation to generate improved vaccines.

In one embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:1 (I3-01 wild type), wherein 1, 2, 3, 4, 5, 6, 7, 8, 9, or all 10 of the following mutations relative to SEQ ID NO:1 are present in the polypeptide: F32Y, H37D/E/K/N/Q/R, F43Q, F168D/E/K/N/Q/R/S/T/Y, K169D/E/N/Q, L173D/E/N/Q/S, A174S, S179D/E, K183D/E, and/or T185D/E/K/N/Q/S. In one non-limiting example of this embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:7-14.

In another embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:2 (O43-38 tetramer wild type), wherein 1, 2, 3, 4, 5, 6, 7, 8, or all 9 of the following mutations relative to SEQ ID NO:2 are present in the polypeptide: M138D/E/K/N/Q/R/S/T, L139D/N/S, A141S, V142R/T, A143S, N146D/E/K/R, R147N, H172D/E/K/N/Q, and/or E173D/K. In one such embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:24-25.

In a further embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:3 (O43-38 trimer wild type), wherein 1, 2, 3, 4, 5, 6, 7, 8, 9, 10, 11, 12, 13, 14, 15, 16, 17, 18, 19, 20, or all 21 of the following mutations relative to SEQ ID NO:3 are present in the polypeptide: R17D/E/K/N/Q/S/T, N19D/E, S20D/E/K/N, V21D/T, V22D/E/Q/S/T, L23D/E/K/N/Q/R/S, A26S, K27N/Q, A30S V31N/S/T, F32R/Y, L33D/E/K/N/Q/R/S/T, H37D/E/K/N/R, F43Q, W167D/E/K/N/Q/R/S/T/Y, F168D/E/K/N/Q/R/S/T/Y, K169D/E/N, L173D/E/N/Q/R/S, A174S, S179D/E/K/N/Q/R, and/or K183D/E/N/Q. In one such embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:26-28.

In one embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:4 (I53 dn5A wild type), wherein 1, 2, 3, 4, 5, 6, 7, 8, 9, or all 10 of the following mutations relative to SEQ ID NO:4 are present in the polypeptide: R17T, W18D/E/K/N/Q/R/S/T/Y, N19E, E21D, L28D/E/K/N/Q/R/S/T/Y, L31D/E/K/N/Q/S/T, K32D/E/N/Q, T118D/E/N/Q/S, L120D/E/K/N/Q/R/S/T, and/or T121D/E/K/N/S. In one such embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:15-23.

In another embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:5 or 6 (hMPV wild type), wherein 1, 2, 3, 4, or all 5, of the following mutations relative to SEQ ID NO:5 or 6 are present in the polypeptide: A107D, V112R, T114E, V118R, and/or G264D. In one such embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:5 or 6, wherein the polypeptide comprises a set of mutations relative to SEQ ID NO:5 or 6 selected from the group consisting of:

(a) T114E+V118R

(b) A107D+V112R+T114E+V118R

(c) A107D+V112R

(d) A107D+V112R+T114E+V118R; and

(e) A107D+V112R+T114E+V118R+G264D.

In various embodiments, the polypeptides comprise or consists of an amino acid sequence at least 90%, 91%, 92%, 93%, 94%, 95%, 96%, 97%, 98%, or 99% identical to the amino acid sequence selected from SEQ ID NO: 29, 31, 33, 35, 37, 39, 41, 43, and 45. In other embodiments, some or all of the residues in parentheses are absent. In further embodiments, some or all of the residues in parentheses are present.

As disclosed herein, the polypeptides of the disclosure may be present in fusion proteins and nanoparticles. Thus, in another embodiment, the disclosure provides fusion proteins comprising:

(a) the polypeptide according to any embodiment of the disclosure; and

(b) a second functional polypeptide.

The second functional polypeptide may have any suitable function, including but not limited to therapeutic polypeptides, diagnostic polypeptides, detectable polypeptides, etc. In one embodiment, the second functional polypeptide comprises an immunogenic portion of a polypeptide antigen. An immunogenic portion of any suitable polypeptide antigen may be used, including but not limited to viral antigens. In one embodiment, the second functional polypeptide comprises an immunogenic portion of an amino acid sequence at least 75%, 80%, 85%, 90%, 91%, 92%, 93%, 94%, 95%, 96%, 97%, 98%, or 99% identical to the amino acid sequence of SEQ ID NO:5 or 6 (hMPV wild type), wherein 1, 2, 3, 4, or all 5, of the following mutations relative to SEQ ID NO:5 or 6 are present in the polypeptide: A107D, V112R, T114E, V118R, and/or G264D;

wherein residues in parentheses are optional and may be present or may be absent in whole or in part. In one embodiment, the second functional polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:5 or 6, wherein the polypeptide comprises a set of mutations relative to SEQ ID NO:5 or 6 selected from the group consisting of:

(a) T114E+V118R

(b) A107D+V112R+T114E+V118R

(c) A107D+V112R

(d) A107D+V112R+T114E+V118R; and

(e) A107D+V112R+T114E+V118R+G264D.

In another embodiment, the fusion protein comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:30, 32, 34, 36, 38, 40, 42, 44, or 46 wherein residues in parentheses are optional and may be present or may be absent in whole or in part. In one embodiment, the residues SGR present in the second optional sequence from the N-terminus of SEQ ID NO: 30, 32, 34, 36, 38, 40, 42, 44, or 46 are present.

In another embodiment, the disclosure provides nanoparticle comprising a plurality of the polypeptides or fusion proteins of any embodiment of combination of embodiments herein. In one embodiment, the nanoparticle comprises

(a) a plurality of polypeptides comprising an amino acid sequence at least 75%, 80%, 85%, 90%, 91%, 92%, 93%, 94%, 95%, 96%, 97%, 98%, or 99% identical to the amino acid sequence of SEQ ID NO:1 (I3-01 wild type), wherein 1, 2, 3, 4, 5, 6, 7, 8, 9, or all 10 of the following mutations relative to SEQ ID NO:1 are present in the polypeptide: F32Y, H37D/E/K/N/Q/R, F43Q, F168D/E/K/N/Q/R/S/T/Y, K169D/E/N/Q, L173D/E/N/Q/S, A174S, S179D/E, K183D/E, and/or T185D/E/K/N/Q/S, or fusion proteins thereof. In one non-limiting example of this embodiment, the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:7-14, or a fusion protein thereof.

In another embodiment, the nanoparticles comprise

(a) a plurality of first polypeptides comprising an amino acid sequence at least 75%, 80%, 85%, 90%, 91%, 92%, 93%, 94%, 95%, 96%, 97%, 98%, or 99% identical to the amino acid sequence of SEQ ID NO:2 (O43-38 tetramer wild type), wherein 1, 2, 3, 4, 5, 6, 7, 8, or all 9 of the following mutations relative to SEQ ID NO:2 are present in the polypeptide: M138D/E/K/N/Q/R/S/T, L139D/N/S, A141S, V142R/T, A143S, N146D/E/K/R, R147N, H172D/E/K/N/Q, and/or E173D/K, or fusion proteins thereof, wherein the plurality of first polypeptides self-interact to form a first multimeric substructure; and

(b) a plurality of second polypeptides comprising an amino acid sequence at least 75%, 80%, 85%, 90%, 91%, 92%, 93%, 94%, 95%, 96%, 97%, 98%, or 99% identical to the amino acid sequence of SEQ ID NO:3 (O43-38 trimer wild type), wherein 1, 2, 3, 4, 5, 6, 7, 8, 9, 10, 11, 12, 13, 14, 15, 16, 17, 18, 19, 20, or all 21 of the following mutations relative to SEQ ID NO:3 are present in the polypeptide: R17D/E/K/N/Q/S/T, N19D/E, S20D/E/K/N, V21D/T, V22D/E/Q/S/T, L23D/E/K/N/Q/R/S, A26S, K27N/Q, A30S V31N/S/T, F32R/Y, L33D/E/K/N/Q/R/S/T, H37D/E/K/N/R, F43Q, W167D/E/K/N/Q/R/S/T/Y, F168D/E/K/N/Q/R/S/T/Y, K169D/E/N, L173D/E/N/Q/R/S, A174S, S179D/E/K/N/Q/R, and/or K183D/E/N/Q, or fusion proteins thereof, wherein the plurality of second polypeptides self-interact to form a second multimeric substructure;

wherein multiple copies of the first multimeric substructure and the second multimeric substructure interact with each other at one or more non-covalent protein-protein interfaces.

In one embodiment, the first polypeptides comprise or consist of an amino acid sequence at least 75%, 80%, 85%, 90%, 91%, 92%, 93%, 94%, 95%, 96%, 97%, 98%, or 99% identical to the amino acid sequence of SEQ ID NO:24-25. In another embodiment, the second polypeptides comprise or consist of an amino acid sequence at least 75%, 80%, 85%, 90%, 91%, 92%, 93%, 94%, 95%, 96%, 97%, 98%, or 99% identical to the amino acid sequence of SEQ ID NO:26-28.

In another embodiment, the nanoparticle comprises

(a) a plurality of first polypeptides comprising an amino acid sequence at least 75%, 80%, 85%, 90%, 91%, 92%, 93%, 94%, 95%, 96%, 97%, 98%, or 99% identical to the amino acid sequence of SEQ ID NO:4 (I53_dn5A), wherein 1, 2, 3, 4, 5, 6, 7, 8, 9, or all 10 of the following mutations relative to SEQ ID NO:4 are present in the polypeptide: R17T, W18D/E/K/N/Q/R/S/T/Y, N19E, E21D, L28D/E/K/N/Q/R/S/T/Y, L31D/E/K/N/Q/S/T, K32D/E/N/Q, T118D/E/N/Q/S, L120D/E/K/N/Q/R/S/T, and/or T121D/E/K/N/S, or fusion proteins thereof, wherein the plurality of first polypeptides self-interact to form a first multimeric substructure; and

(b) a plurality of second polypeptides comprising or consisting of SEQ ID NO:47 that self-interact to form a second multimeric substructure;

wherein multiple copies of the first multimeric substructure and the second multimeric substructure interact with each other at one or more non-covalent protein-protein interfaces. In one such embodiment, the first polypeptides comprise or consist of an amino acid sequence at least 75%, 80%, 85%, 90%, 91%, 92%, 93%, 94%, 95%, 96%, 97%, 98%, or 99% identical to the amino acid sequence of SEQ ID NO:15-23.

(I53_dn5B; SEQ ID NO: 47)
(M)EEAELAYLLGELAYKLGEYRIAIRAYRIALKRDPNNAEAWYNLGNAYY
KQGRYREAIEYYQKALELDPNNAEAWYNLGNAYYERGEYEEAIEYYRKALR
LDPNNADAMQNLLNAKMREE

In all of these embodiments of nanoparticles, the plurality of component polypeptides may comprise one or more fusion proteins. When one or more fusion proteins are present, one or more of the fusion proteins comprise a second functional polypeptide as described above. Such second functional polypeptides may include but not limited to an immunogenic portion of a polypeptide antigen, wherein the polypeptide antigen includes but is not limited to the polypeptide comprising an immunogenic portion of an amino acid sequence at least 75%, 80%, 85%, 90%, 91%, 92%, 93%, 94%, 95%, 96%, 97%, 98%, or 99% identical to the amino acid sequence of SEQ ID NO:5 or 6 (hMPV wild type), wherein 1, 2, 3, 4, or all 5, of the following mutations relative to SEQ ID NO:5 or 6 are present in the polypeptide: A107D, V112R, T114E, V118R, and/or G264D;

wherein residues in parentheses are optional and may be present or may be absent in whole or in part. In one embodiment, the second functional polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:5 or 6, wherein the polypeptide comprises a set of mutations relative to SEQ ID NO:5 or 6 selected from the group consisting of:

(a) T114E+V118R

(b) A107D+V112R+T114E+V118R

(c) A107D+V112R

(d) A107D+V112R+T114E+V118R; and

(e) A107D+V112R+T114E+V118R+G264D, or

wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:29, 31, 33, 35, 37, 39, 41, 43, or 45.

In another embodiment, the disclosure provides compositions comprising a plurality of nanoparticles according to any embodiment or combination of embodiments described herein. The compositions may be used for any of the uses described herein, including but not limited for use as vaccines when loaded with immunogenic portions of a polypeptide antigen.

In another embodiment, the disclosure provides a synthetic (“degreased”) nanoparticle, comprising a cryptic transmembrane domain, wherein one or more of the hydrophobic amino acids of the cryptic transmembrane domain have been substituted with a polar amino acid. In one embodiment, the amino acid substitution is within a 19-residue sliding window for transmembrane insertion potential (dG_ins); windows of dG_ins less than or equal to +2.7 kcal/mol are confirmed to be local minima within +/−9 residues, and the cutoff of +2.7 kcal/mol is the signature of the cryptic transmembrane domain. In another embodiment, the synthetic nanoparticle comprises a polypeptide comprising the amino acid sequence of SEQ ID NO:13.

In one embodiment, the synthetic nanoparticle is a polypeptide. In other embodiments, the synthetic nanoparticle comprises a signal peptide and/or a tag. In another embodiment, the synthetic nanoparticle comprises a one-component or homomeric nanoparticle. In one such embodiment, the synthetic nanoparticle comprises an expressed sequence as shown and described herein.

In another embodiment, the synthetic nanoparticle comprises variant I3-01 amino acid sequences. In one such embodiment, the synthetic nanoparticle comprises a polar amino acid substitution at position 25, position, 35, position 171, position 177, or position 180, or at any two or more combinations of those positions. In a further embodiment, the synthetic nanoparticle further comprises an agent to be secreted (“secreted agent”). In one such embodiment, the secreted agent is selected from:
a) a polypeptide;
b) a payload; and
c) an antigen displayed on the exterior of the synthetic nanoparticle

In one embodiment, the polypeptide comprises an antigen an antigen immunogenic portion of an antigen. In another embodiment, the antigen immunogen or immunogenic is of viral origin. In one embodiment, the virus is human metapneumo virus (hMPV).

In another embodiment, the synthetic nanoparticle comprises a two-component nanoparticle. In one such embodiment, the synthetic nanoparticle comprises a trimer, a tetramer, or a pentamer. In another embodiment, the synthetic nanoparticle is selected from: I53_dn5, O43-38, and I53-50. In another embodiment, the synthetic nanoparticle is 153_dn5 and wherein the pentameric subunit I53_dn5A of the synthetic nanoparticle comprises a polar amino acid substitution at least one of position 16, position 29, position 116, position 118, or position 119, or at any two or more combinations of those positions.

In one embodiment the synthetic nanoparticle is O43-38 and wherein the tetrameric subunit O43-38tet of the synthetic nanoparticle comprises a polar amino acid substitution at position 29, position 141, position 19, position 21, or position 31, or at any two or more combinations of those positions.

In another aspect the disclosure provides nucleic acids encoding the polypeptide, fusion proteins, or nanoparticles of any embodiment or combination of embodiments of the disclosure. The nucleic acid sequence may comprise single stranded or double stranded RNA or DNA in genomic or cDNA form, mRNA, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptide, fusion protein, nanoparticle, composition, nucleic acid, and/or expression vector (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another embodiment, the disclosure provides pharmaceutical compositions comprising:

(a) the polypeptide, fusion protein, nanoparticle, composition, nucleic acid, expression vector, and/or host cell of any embodiment or combination of embodiments herein; and

(b) a pharmaceutically acceptable carrier.

The pharmaceutical compositions of the disclosure can be used, for example, in the methods of the disclosure described below. The pharmaceutical composition may comprise in addition to the polypeptide or other active agent of the disclosure (a) a lyoprotectant; (b) a surfactant; (c) a bulking agent; (d) a tonicity adjusting agent; (e) a stabilizer; (0 a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The pharmaceutical composition and the compositions may further comprise one or more other active agents suitable for an intended use.

In another aspect, the disclosure provides methods of delivering a secreted agent from a cell, comprising administering or admixing the cell with the nucleic acid molecule and/or the expression vector of any embodiment or combination of embodiments herein and secreting the nanoparticle or synthetic nanoparticle.

In another aspect, the disclosure provides vaccines comprising the nanoparticle, composition, pharmaceutical composition, synthetic nanoparticle, nucleic acid, expression vector, and/or cell of any embodiment or combination of embodiments herein.

In a further aspect, the disclosure provides methods to vaccinate a subject against a virus, the method comprising administering the nanoparticle, composition, pharmaceutical composition, synthetic nanoparticle(s) or the vaccine(s) described herein to the subject. The subject may be any suitable subject, including but not limited to a mammalian subject such as a human subject. In one embodiment, the method comprises

(a) obtaining the nanoparticle, composition, pharmaceutical composition, synthetic nanoparticles, the compositions, or the vaccines described herein; and,

(b) administering the synthetic nanoparticles, the compositions, or the vaccines described herein to the subject.

In another embodiment, the administration elicits an immune response in the subject, such that the subject is protected against infection.

The disclosure also provides kits, comprising one or more components selected from the group consisting of the polypeptide, fusion protein, nanoparticle, composition, synthetic nanoparticle(s), the nucleic acid molecule(s), the expression vector(s), the cell(s), the composition(s),or the vaccine(s) described herein.

In another aspect, the disclosure provides computer-implemented methods for designing a secreted peptide, using any suitable methods as described herein. In one embodiment, the methods comprise:

generating a 3D structure of a protein of interest with a 19-residue sliding window for transmembrane insertion potential (dG_ins);

wherein windows of dG_ins less than or equal to +2.7 kcal/mol are confirmed to be local minima within +/−9 residues, and the cutoff of +2.7 kcal/mol is the signature of a cryptic transmembrane domain;

designing one or more peptide sequences based on the generated 3D structure and predicting mutations at each position within that domain, wherein allowed residues are all polar, excluding histidine, such that the final allowable residues are amino acids D, E, K, R, Q, N, S, T, Y; and side chains of other residues within an 8-Angstrom shell are allowed to adopt different rotamers (“repack” to one of skill in the art) but not mutate to other residues (“design” to one of skill in the art).

In one embodiment, for each mutation or set of mutations, the score of the overall energy of the structure is generated and wherein

(a) if the new score is higher than the original score by a threshold amount of 15 REU (dscore), the degreaser variant is discarded and not further evaluated; or

(b) if the new score is within the tolerance, but the change in dG_ins is less than +0.27 kcal/mol (ddG_ins), the mutation placed at that position is rejected and disallowed at that position, and the position is subjected to mutation again; or

(c) if the new score is within the tolerance and the ddG_ins is greater than +0.27 kcal/mol, the mutation is accepted, the structure is optionally output, and the metrics of that mutation are written to the final output file.

Each position within such a domain is thusly evaluated and mutated, and each domain within the sequence is thusly evaluated and mutated. The final outputs may be written to the end of the output structure file, examples of which are shown in Tables 1 and 2.

### EXAMPLES

Many proteins, including but not limited to viral glycoprotein antigens, must be expressed as secreted proteins in eukaryotic cells. This requirement can derive from many different causes, including but not limited to a requirement for post-translational modifications including but not limited to N-linked glycosylation, disulfide bond formation, etc. However, the yield of secreted protein from eukaryotic cells varies widely for reasons that are not fully understood by those of skill in the art, and some proteins altogether fail to secrete at appreciable levels. Here we describe the identification of cryptic transmembrane domains in a variety of protein sequences that accounts for their poor secretion from eukaryotic cells. We further describe that eliminating these cryptic transmembrane domains through the mutation of hydrophobic residues to polar residues improves the yield of secreted protein. We disclose a general computational method for the identification of cryptic transmembrane domains and their removal through mutation without disrupting a protein's overall structure. We further disclose examples of both designed nanoparticle proteins and viral glycoprotein antigens whose secretion was improved using the method.

A general computational method to predict putative transmembrane domains and redesign them. Across all domains of life, membrane proteins are interpreted by a protein complex known as the translocon. In eukaryotes, Sec61 and its associated chaperones recognize proteins destined for the secretory pathway, plasma membrane resident and extracellularly secreted, via an amino-terminal signal peptide. As the protein is translated, segments of high hydrophobicity partition into the ER membrane.

We have found that several designed protein nanoparticle components, although solubly and stably expressed in bacterial systems, were incompatible with eukaryotic secretion. This differential expression in eukaryotic cells was not correlated with bacterial expression levels. Initial attempts to rationally redesign sequences by structural examination did not afford secretable nanoparticle components. Thus, a model-guided design method was needed in order to improve secretion of these proteins.

A general computational method for designing protein sequences for improved secretion from eukaryotic cells. We wrote code that describes the amino acid- and position-specific contribution to transmembrane insertion at each position within a given segment of a protein. We integrated this code into the Rosetta™ macromolecular modeling and design suite to enable simultaneous design away from high hydrophobicity and toward native stability, such that mutations introduced to remove cryptic transmembrane segments do not destabilize the protein's native structure. We refer to this design protocol as the Degreaser™. Initial input parameters for the degreaser were empirically determined by visual inspection of a range of outputs, with the intention of minimally perturbing the existing designed interfaces.
Characterization of Degreaser™ variants. The Degreaser predicted several variants for each protein input. Each variant generated had increased predicted transmembrane insertion potential, confirming the intended behavior of the Degreaser. Several variants were generated for each input structure, which were then visually inspected. The initial set of proteins examined were: I3-01, a one-component icosahedral particle that was designed using the trimeric 1wa3-wt protein as a starting point,; I53-dn5A, the pentameric component of a two-component icosahedral nanoparticle, designed from PDB 2jfb, and the tetrameric and trimeric components of the two-component octahedral nanoparticle O43-38, designed starting from PDBs 1e4c and 1wa3, respectively. 1wa3-wt was solubly secreted from HEK293F suspension cells when appended to an IgK secretion signal; the nanoparticle components were not appreciably secreted.

### A. Construction of a Rosetta™ “Mover” to Identify, Perturb, and Evaluate Candidate Variants, Working Name “Degreaser™”

a. Definition: “Degreaser™” refers to the program that was written and compiled in C++ as part of the Rosetta™ macromolecular modeling package in order to use standard Rosetta features such as PDB handling and other scoring metrics.
b. Definition: a “degreased” protein sequence can be said to have been evaluated by the Degreaser, and to have been experimentally validated to have improved secretion from a eukaryotic cell. Candidates that were evaluated by the degreaser but not experimentally evaluated for improved secretion from a eukaryotic cell, or other candidates that were not evaluated, can be called “not degreased.”
c. Definition: a “degreaser variant” refers to a candidate output from the degreaser before it is/was classified as “degreased” or “not degreased.”
d. The core of the code is briefly outlined here. The input 3D structure of interest is evaluated with a 19-residue sliding window for transmembrane insertion potential (dG_ins). Windows of dG_ins less than or equal to +2.7 kcal/mol are confirmed to be local minima within +/−9 residues, and the cutoff of +2.7 kcal/mol is the signature of a ‘cryptic transmembrane domain.’ Once all such domains are recognized, the program uses the Rosetta™ Packer to make mutations at each position within that domain. The allowed residues were all polar, excluding histidine, such that the final allowable residues were “DEKRQNSTY.” (SEQ ID NO: 62) After the Packer makes a change to a residue in the domain, side chains of other residues within an 8-Angstrom shell were allowed to adopt different rotamers (“repack” to one of skill in the art) but not mutate to other residues (“design” to one of skill in the art). For each mutation or set of mutations, the Rosetta score, or overall energy of the structure, is evaluated, as well as the new dG_ins. If the new score was higher than the original score by a threshold amount of 15 REU (dscore), the degreaser variant is discarded and not further evaluated. If the new score is within the tolerance, but the change in dG_ins is less than +0.27 kcal/mol (ddG_ins), the mutation placed at that position is rejected and disallowed at that position, and the position is subjected to mutation again. If the new score is within the tolerance and the ddG_ins was greater than +0.27 kcal/mol, the mutation is accepted, the structure is optionally output, and the metrics of that mutation are written to the final output file. Each position within such a domain is thusly evaluated and mutated, and each domain within the sequence is thusly evaluated and mutated. The final outputs are written to the end of the output structure file, examples of which are shown in Tables 1 and 2.
e. All degreaser variants were inspected by looking at the mutant's 3D structure in PyMol. Some outputs that appeared unrealistic as would be known to one of skill in the art, such as the incorporation of charged residues into the hydrophobic core of a protein, were removed from the variant list. Furthermore, only a select number of candidates for each scaffold were chosen for experimental evaluation.

### B. Expression and Screening of Nanoparticle Proteins and Degreaser Variants of Nanoparticle Proteins

Definition: “pCMV” refers to a pcDNA3.1-based expression vector.

Definition: “IgK signal peptide” refers to the amino acid sequence “METDTLLLWVLLLWVPGSTGD (SEQ ID NO: 48)” and “IgK-mini-FLAG” refers to the amino acid sequence “METDTLLLWVLLLWVPGSTGDYKDEK (SEQ ID NO: 49)”.

Definition: “His tag” refers to the amino acid sequence “HHHHHH (SEQ ID NO: 50)”.

Definition: “myc tag” refers to the amino acid sequence “EQKLISEEDL (SEQ ID NO: 51)”.

Unless otherwise specified, all constructs experimentally evaluated for secretion from a eukaryotic cell contain an IgK signal peptide or IgK-mini-FLAG at the amino terminus and a myc tag immediately followed by a His tag at the carboxy terminus.

For I3-01, degreaser variants were generated by two-round PCR amplification. In brief, primers annealing to 5′ and 3′ regions of the multiple cloning site in the pCMV expression vector encoding I3-01 were designed to be universal. Then, for each variant, a primer was designed to incorporate the mutation(s) of interest. The first round of amplification generated a 100- to 200-base pair “megaprimer,” which was then used in a second round of amplification to generate a linear, double-stranded DNA fragment encoding the degreaser variant of interest. These mutation-bearing DNA sequences were ligated by Gibson assembly into PCR-linearized vector. All sequences were validated by forward and reverse sequencing reads upstream and downstream of the gene of interest, respectively.

For other degreaser variants, human codon-optimized sequences were synthesized by Genscript or IDT, then cloned into existing vectors by Gibson assembly. hMPV F proteins and degreased hMPV F protein variants were synthesized with the hMPV F native signal peptide rather than “IgK” or “IgK-mini-FLAG.”

Plasmids of pCMV harboring degreaser variants were transformed into NEB 5-alpha high-efficiency chemically competent cells per the manufacturer's instructions. Cultures were inoculated in TB or LB media containing suitable antibiotics. Plasmids were prepared with Qiagen Plasmid Miniprep kits according to the manufacturer's instructions.

Purified plasmids were transfected into HEK293F suspension cell culture using PEI, per the manufacturer's instructions. Cells were harvested three, four, or five days after transfection. Medium was separated from cells by centrifugation at 1,500×g.

### C. Fractionation and Western Blotting of Cell Culture

Definition: “anti” refers to an antibody raised against a particular epitope; e.g. an “anti-myc” antibody binds to myc-tagged polypeptides.

Definition: “TBS” refers to Tris-buffered saline, and is pH 8.0 unless otherwise specified.

Cell and supernatant fractions were treated with 0.5% Triton-X 100 containing >2.5 U/uL of Benzonase™ nuclease for 10 minutes at 37° C. Samples were then diluted for SDS-PAGE into 50 mM Tris pH 6.8, 2% SDS, 10% glycerol, and at least 1 mM DTT. Samples in SDS buffer were incubated at 95° C. for five minutes before being loaded onto pre-cast 4-20% Criterion™ gels (BIO-RAD). Gels were run at 250V for 26 minutes, then transferred onto nitrocellulose membranes (BIO-RAD) from a Trans-blot Turbo kit according to manufacturer's instructions. Transferred membranes were optionally stained with Ponceau™ S per manufacturer's instructions. Membranes were then blocked with 3% blotting-grade blocker (BIO-RAD) in TBS supplemented with 0.1% Tween-20. Anti-myc antibody, mouse monoclonal (Cell Signaling Technologies), was diluted 1 in 20,000 in the same blocking buffer and incubated with the membrane. After incubation and wash with TBS with 0.1% Tween-20, anti-mouse IgG HRP-conjugate was diluted 1 in 20,000, and StrepTactin™ anti-ladder was diluted 1 in 50,000 in fresh blocking buffer and incubated with the membrane. After incubation and wash, the membranes were visualized with Clarity ECL substrate (BIO-RAD) per the manufacturer's instructions on a BIO-RAD GelDoc™ Imager.

Western blotting was the main assay used to detect improvements to secretion levels. Looking at the ratio of secreted protein to total protein controlled for potential expression differences among variants, although those differences were minimal, likely due to there being only one mutation per variant. Semi-quantitative measurements could be made using ImageJ software to analyze the raw blot images by densitometry. For each scaffold tested; that is, I3-01, O43-38 tetramer, O43-38 trimer, and I53-dn5A pentamer, at least one variant significantly (>50%) improved secretion yields. Each degreased variant is not necessarily the variant that had the highest dG_ins; in those cases, the poor secretion of the variants with the highest dG_ins could be due to destabilization of the protein or other unforeseen effects.

### D. Purification of Protein from Cell Culture Supernatant

After cell culture supernatant was filtered through a 0.45 μm filter, 40 uL of Ni-NTA slurry was added to 1 mL of supernatant. This mixture was incubated, then resin was sedimented by centrifugation. Three washes of increasing imidazole concentration (10 mM, 20 mM, and 50 mM) were used to remove unwanted contaminants. Finally, the protein of interest was eluted with 500 mM imidazole in a Tris buffer.

Later constructs were purified with Ni Excel™ Sepharose (GE Healthcare) according to manufacturer's instructions.

Purification of protein from cell culture supernatant also served to increase the concentration of protein in the samples analyzed. The transition was made from Ni-NTA resin to Ni Excel™ Sepharose after poor yields were obtained with Ni-NTA, which was attributed to EDTA present in cell culture media that may strip Ni ions from the resin.

### E. Transmission Electron Microscopy of Protein Nanoparticles

Samples were prepared for negative stain EM by diluting to 0.05-0.075 mg/mL using a Tris-based buffer, and 6.0 μL was incubated on a glow-discharged, copper, carbon-coated grid for 1 min before quickly immersing the grid in a 60 μL drop of water. The water was blotted off within seconds by Whatman™ No. 1 filter paper, and the grid was immediately dipped into a 6.0 μL drop of stain (2% w/v uranyl formate). The stain was immediately blotted away and within seconds the grid was dipped into another 6.0 μL drop of stain, which was left on the grid for 30 seconds. At the end of this time, the stain was blotted dry and allowed to air dry for 5 minutes prior to imaging. Images were recorded on a FEI Morgagni 268 transmission electron microscope equipped with a Gatan US4000 CCD camera, using Leginon™ software for data collection at a nominal magnification of 22,000× at a defocus range comprised between −1 um and −4 um.

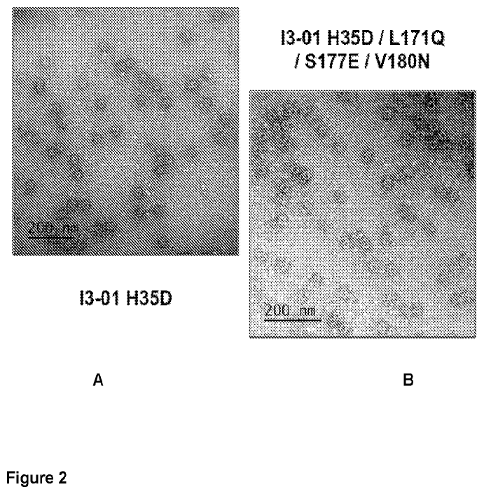

TEM was the primary assay used to determine preservation of original protein architecture, especially in the case of I3-01, as it is secreted as a full nanoparticle. This method was preferable to other assays as it is a fast and definitive readout for assembly versus no assembly. As shown in FIG. 2, the protein still forms icosahedral nanoparticles that can be visualized by TEM, demonstrating that the mutations made to the protein do not significantly affect protein structure or assembly.

### F. In Vitro Assembly of Protein Nanoparticles

For secreted individual nanoparticle components, assembly competency could not be directly assessed by TEM of those proteins, as was possible with I3-01. Therefore, purified components from cell culture supernatant were mixed at a 1:1 ratio with the appropriate second component in order to form nanoparticle assemblies. The second component was typically produced in bacterial culture as previously described. Assembly reactions were then purified by size-exclusion chromatography. Most variants demonstrated good assembly competency. An exception was the assembly of degreased O43-38 tetramer with degreased O43-38 trimer, indicating that the mutations made to both components of this architecture interfered with assembly of the nanoparticle.

### G. ELISA Determination of Protein in Cell Culture Supernatants

Filtered supernatants containing degreaser variants were bound to Nunc MaxiSorp™ 96-well plates in a two-fold dilution series. Antibodies specific to a tag or known epitope of interest were first applied, followed by a secondary anti-human antibody conjugated to HRP. For nanoparticle proteins and degreased nanoparticle proteins, protein yield was determined colorimetrically using the substrate TMB and absorbances were collected at 450 nm. For hMPV F proteins and degreased hMPV F protein variants, protein yield was determined colorimetrically using the substrate ABTS and absorbances were collected at 405 nm.

Design and experimental evaluation of degreased hMPV F genetically fused to nanoparticle proteins. In addition to proteins in which cryptic transmembrane domains have been introduced by mutation, computational design, or directed evolution, some naturally occurring proteins also contain cryptic transmembrane domains. For example, many viral fusion glycoproteins have long stretches of hydrophobic amino acids that contain the “fusion peptides” the glycoproteins insert into host cell membranes during the membrane fusion process. One non-limiting example of such a protein is hMPV F (e.g., the Arg/2/02 isolate; Genbank ABD27846.1), which has three strongly predicted transmembrane domains at positions 103-125, 256-278, and 514-530. Only the region from residues 514-530 is known to traverse the viral membrane; residues 103-125 comprise the fusion peptide, while residues 256-278 have not been previously reported to interact with membranes. We used the degreaser to make several degreased variants of prefusion hMPV F (“115-BV”; Battles et al., Nat. Comm. 2017) and expressed them as genetic fusions to the nanoparticle components I53-50 and I53_dn5. We found that several of these variants, and in particular hMPV F-50A_14, which contains four degreaser mutations, secreted more efficiently from mammalian cells than corresponding non-degreased constructs. This non-limiting example demonstrates that the degreaser protocol may be used to improve the secretion of naturally occurring proteins that contain cryptic transmembrane domains.

### REFERENCES

1. Air G M. Influenza virus antigenicity and broadly neutralizing epitopes. Curr Opin Virol. 2015; 11:113-21.
2. Gaschen B, Taylor J, Yusim K, Foley B, Gao F, Lang D, Novitsky V, Haynes B, Hahn B H, Bhattacharya T, Korber B. Diversity considerations in HIV-1 vaccine selection. Science (80-). 2002; 296 (5577):2354-60.
3. Draper S J, Sack B K, King C R, Nielsen C M, Rayner J C, Higgins M K, Long C A, Seder R A. Malaria Vaccines: Recent Advances and New Horizons. Cell Host Microbe. 2018; 24 (1):43-56.
4. Graham B S, Gilman M S A, McLellan J S. Structure-Based Vaccine Antigen Design. Annu Rev Med. 2019; 70 (1):91-104.
5. King N P, Bale J B, Sheffler W, McNamara D E, Gonen S, Gonen T, Yeates T O, Baker D. Accurate design of co-assembling multi-component protein nanomaterials. Nature. 2014; 510 (7503):103-8.
6. Zhao L, Seth A, Wibowo N, Zhao C X, Mitter N, Yu C, Middelberg A P J. Nanoparticle vaccines. Vaccine. 2014; 32 (3):327-37.
7. López-Sagaseta J, Malito E, Rappuoli R, Bottomley M J. Self-assembling protein nanoparticles in the design of vaccines. Comput Struct Biotechnol J. 2016; 14:58-68.
8. Kanekiyo M, Wei C J, Yassine H M, McTamney P M, Boyington J C, Whittle J R R, Rao S S, Kong W P, Wang L, Nabel G J. Self-assembling influenza nanoparticle vaccines elicit broadly neutralizing H1N1 antibodies. Nature. 2013; 499 (7456):102-6.
9. Huang P S, Boyken S E, Baker D. The coming of age of de novo protein design. Nature. 2016; 537 (7620):320-7.
10. Marcandalli J, Fiala B, Ols S, Perotti M, de van der Schueren W, Snijder J, Hodge E, Benhaim M, Ravichandran R, Carter L, Sheffler W, Brunner L, Lawrenz M, Dubois P, Lanzavecchia A, Sallusto F, Lee K K, Veesler D, Correnti C E, Stewart L J, Baker D, Loré K, Perez L, King N P. Induction of Potent Neutralizing Antibody Responses by a Designed Protein Nanoparticle Vaccine for Respiratory Syncytial Virus. Cell. 2019; 176 (6):1420-1431.e17.
11. Butterfield G L, Lajoie M J, Gustafson H H, Sellers D L, Nattermann U, Ellis D, Bale J B, Ke S, Lenz G H, Yehdego A, Ravichandran R, Pun S H, King N P, Baker D. Evolution of a designed protein assembly encapsulating its own RNA genome. Nature. 2017; 552 (7685):415-20.
12. Pardi N, Hogan M J, Porter F W, Weissman D. mRNA vaccines-a new era in vaccinology. Nat Rev Drug Discov. 2018; 17 (4):261-79.
13. Nishikawa I, Nakajima Y, Ito M, Fukuchi S, Homma K, Nishikawa K. Computational prediction of O-linked glycosylation sites that preferentially map on intrinsically disordered regions of extracellular proteins. Int J Mol Sci. 2010; 11 (12):4991-5008.
14. Denks K, Vogt A, Sachelaru I, Petriman N A, Kudva R, Koch H G. The Sec translocon mediated protein transport in prokaryotes and eukaryotes. Vol. 31, Molecular Membrane Biology. 2014. p. 58-84.
15. Hessa T, Meindl-Beinker N M, Bernsel A, Kim H, Sato Y, Lerch-Bader M, Nilsson I, White S H, Von Heijne G. Molecular code for transmembrane-helix recognition by the Sec61 translocon. Nature. 2007 Dec. 13; 450 (7172):1026-30.
16. Cherf G M, Cochran J R. Applications of yeast surface display for protein engineering. Methods Mol Biol. 2015; 1319:155-75.
17. Boder E T, Wittrup K D. Yeast surface display for directed evolution of protein expression, affinity, and stability. Methods Enzymol. 2000; 328 (1999):430-44.
18. Bhaskar A, Chawla M, Mehta M, Parikh P, Chandra P, Bhave D, Kumar D, Carroll K S, Singh A. Reengineering Redox Sensitive GFP to Measure Mycothiol Redox Potential of Mycobacterium tuberculosis during Infection. PLoS Pathog. 2014; 10 (1).
19. Plotkin S. Vaccines: past, present and future Early successes. Nat Med. 2005; 11 (4):5-11.
20. Patil S U, Shreffler W G. Novel vaccines: Technology and development. J Allergy Clin Immunol. 2019; 143 (3):844-51.
21. Pulendran B, Ahmed R. Immunological mechanisms of vaccination. Nat Immunol. 2011; 12 (6):509-17.
22. Delany I, Rappuoli R, De Gregorio E. Vaccines for the 21st century. EMBO Mol Med. 2014; 6 (6):708-20.
23. Morein B, Simons K. Subunit vaccines against enveloped viruses: virosomes, micelles and other protein complexes. Vaccine. 1985; 3 (2):83-93.
24. Moyle P M, Toth I. Modern Subunit Vaccines: Development, Components, and Research Opportunities. ChemMedChem. 2013 March; 8 (3):360-76.
25. Vartak A, Sucheck S J. Recent advances in subunit vaccine carriers. Vaccines. 2016; 4 (2): 1-18.
26. Burton D R, Hangartner L. Broadly Neutralizing Antibodies to HIV and Their Role in Vaccine Design. Annu Rev Immunol. 2016; 34 (1):635-59.
27. Pica N, Palese P. Toward a Universal Influenza Virus Vaccine: Prospects and Challenges. Annu Rev Med. 2013; 64 (1):189-202.
28. Kanekiyo M, Joyce M G, Gillespie R A, Gallagher J R, Andrews S F, Yassine H M, Wheatley A K, Fisher B E, Ambrozak D R, Creanga A, Leung K, Yang E S, Boyoglu-Barnum S, Georgiev I S, Tsybovsky Y, Prabhakaran M S, Andersen H, Kong W P, Baxa U, Zephir K L, Ledgerwood J E, Koup R A, Kwong P D, Harris A K, McDermott A B, Mascola J R, Graham B S. Mosaic nanoparticle display of diverse influenza virus hemagglutinins elicits broad B cell responses. Nat Immunol. 2019; 20 (3):362-72.
29. Ra J-S, Shin H-H, Kang S, Do Y. Lumazine synthase protein cage nanoparticles as antigen delivery nanoplatforms for dendritic cell-based vaccine development. Clin Exp Vaccine Res. 2014; 3 (2):227.
30. Harcus T E, Gluckman M, Pontzer H, Raichlen D A, Marlowe F W, Siegfried W R, Macdonald I A W, Call J, Fischer J, Stryjewski K F, Quader S, Sorenson M D, Boogert N, Davies N, Flower T, Jamie G, Magrath R, Rendall D, Ruxton G, Sorensen M, Wood B, David C, Bale J B, Gonen S, Liu Y, Sheffler W, Ellis D, Thomas C, Cascio D, Yeates T O, Gonen T, King N P, Baker D. Accurate design of megadalton-scale two-component icosahedral protein complexes. Science (80-). 2016; 353 (6297):389-95.
31. Hsia Y, Bale J B, Gonen S, Shi D, Sheffler W, Fong K K, Nattermann U, Xu C, Huang P S, Ravichandran R, Yi S, Davis T N, Gonen T, King N P, Baker D. Design of a hyperstable 60-subunit protein icosahedron. Nature. 2016 Jul. 15; 535 (7610):136-9.
32. Zandi R, Reguera D, Bruinsma R F, Gelbart W M, Rudnick J. Origin of icosahedral symmetry in viruses. Proc Natl Acad Sci USA. 2004; 101 (44):15556-60.
33. Braakman I, Hebert D N. Protein Folding in the Endoplasmic Reticulum. Compr Biotechnol Second Ed. 2011; 1:217-27.
34. Nyathi Y, Wilkinson B M, Pool M R. Co-translational targeting and translocation of proteins to the endoplasmic reticulum. Biochim Biophys Acta—Mol Cell Res. 2013; 1833 (11):2392-402.
35. Cymer F, Von Heijne G, White S H. Mechanisms of integral membrane protein insertion and folding. J Mol Biol. 2015; 427 (5):999-1022.
36. Hessa T, Kim H, Bihlmaier K, Lundin C, Boekel J, Andersson H, Nilsson I M, White S H, Von Heijne G. Recognition of transmembrane helices by the endoplasmic reticulum translocon. Nature. 2005; 433 (7024):377-81.
37. Thomas G. Furin at the cutting edge: From protein traffic to embryogenesis and disease. Nat Rev Mol Cell Biol. 2002; 3 (10):753-66.
38. Remade A G, Shiryaev S A, Oh E S, Cieplak P, Srinivasan A, Wei G, Liddington R C, Ratnikov B I, Parent A, Desjardins R, Day R, Smith J W, Lebl M, Strongin A Y. Substrate cleavage analysis of furin and related proprotein convertases: A comparative study. J Biol Chem. 2008; 283 (30):20897-906.
39. Ryan M D, King A M Q, Thomas G P. Cleavage of foot-and-mouth disease virus polyprotein is mediated by residues located within a 19 amino acid sequence. J Gen Virol. 1991; 72 (11):2727-32.
40. Donnelly M L L, Luke G, Mehrotra A, Li X, Hughes L E, Gani D, Ryan M D. Analysis of the aphthovirus 2A/2B polyprotein “cleavage” mechanism indicates not a proteolytic reaction, but a novel translational effect: A putative ribosomal “skip.” J Gen Virol. 2001; 82 (5):1013-25.
41. De Felipe P, Luke G A, Hughes L E, Gani D, Halpin C, Ryan M D. E unum pluribus: Multiple proteins from a self-processing polyprotein. Trends Biotechnol. 2006; 24 (2):68-75.
42. Liu Z, Chen O, Wall J B J, Zheng M, Zhou Y, Wang L, Ruth Vaseghi H, Qian L, Liu J. Systematic comparison of 2A peptides for cloning multi-genes in a polycistronic vector. Sci Rep. 2017; 7 (1):1-9.
43. Pagny S, Cabanes-Macheteau M, Gillikin J W, Leborgne-Castel N, Lerouge P, Boston R S, Faye L, Gomord V. Protein recycling from the Golgi apparatus to the endoplasmic reticulum in plants and its minor contribution to calreticulin retention. Plant Cell. 2000; 12 (5):739-55.
44. Zakeri B, Fierer J O, Celik E, Chittock E C, Schwarz-Linek U, Moy V T, Howarth M. Peptide tag forming a rapid covalent bond to a protein, through engineering a bacterial adhesin. Proc Natl Acad Sci USA. 2012; 109 (12).
45. Pinder C L, Kratochvil S, Cizmeci D, Muir L, Guo Y, Shattock R J, McKay P F. Isolation and Characterization of Antigen-Specific Plasmablasts Using a Novel Flow Cytometry-Based Ig Capture Assay. J Immunol. 2017; 199 (12):4180-8.
46. Chuang K H, Hsieh Y C, Chiang I S, Chuang C H, Kao C H, Cheng T C, Wang Y T, Lin W W, Chen B M, Roffler S R, Huang M Y, Cheng T L. High-throughput sorting of the highest producing cell via a transiently protein-anchored system. PLoS One. 2014; 9 (7): 1-7.

TABLE 1
A curated list of I3-01 degreaser variants that were experimentally
characterized.

| | | | | | |
|---|---|---|---|---|---|
| DEGREASER: 101 | KLNFELGIPVIFGVLNCDK | 3.836 | xxxxx.xx | 1.937 | x.xxx |
| (SEQ ID NO: 52) | T116N, L118D, T119K | | | | |
| DEGREASER: 101 | KLNFELGIPVIFGVLNCLT | 2.862 | −1381.41 | 0.964 | 12.721 |
| (SEQ ID NO: 53) | T116N | | | | |
| DEGREASER: 101 | KLNFELGIPVIFGVLTCDT | 2.612 | −1386.55 | 0.714 | 7.578 |
| (SEQ ID NO: 54) | L118D | | | | |
| DEGREASER: 101 | KLNFELGIPVIFGVLTCLK | 2.170 | −1384.63 | 0.272 | 9.499 |
| (SEQ ID NO: 55) | T119K | 1.897 | −1394.13 | 0.000 | 0.000 |
| DEGREASER: 101 | KLNFELGIPVIFGVLTCLT | | | | |
| (SEQ ID NO: 56) | | | | | |

Index refers to the amino acid position of the first amino acid in the potential transmembrane domain. Sequence refers to the sequence of the domain or variant at that position. dG_ins is the predicted transmembrane potential, with lower numbers more likely to be poorly secreting. Score is the Rosetta-calculated energy. ddG_ins and dscore are the differences in dG_ins and score relative to the unperturbed structure, respectively. Note that some variants were manually designed, and thus have no Rosetta score evaluated for them, though their dG_ins can still be calculated.

TABLE 2
A curated list of I53-dn5 pentamer degreaser variants that were experimentally
characterized.

| I53-dn5A degreaser | | | | | |
|---|---|---|---|---|---|
| result output | | | | | |
| index mutants | sequence | dG_ins | score | ddG_ins | dscore |
| DEGREASER: 14 | ARWNAEIILALVEGALKRL | 2.812 | −1385.69 | 2.033 | 8.442 |
| (SEQ ID NO: 57) | L26E | | | | |
| DEGREASER: 14 | ARENAEIILALVLGALKRL | 2.469 | −1392.97 | 1.690 | 1.160 |
| (SEQ ID NO: 58) | W16E | | | | |
| DEGREASER: 14 | ARWNAEIILALVLGANKRL | 2.019 | xxxxx.xx | 1.240 | x.xxx |
| (SEQ ID NO: 59) | L29N | | | | |
| DEGREASER: 14 | ARWNAEIILALVLGALERL | 1.128 | −1390.51 | 0.349 | 3.625 |
| (SEQ ID NO: 60) | K30E | | | | |
| DEGREASER: 14 | ARWNAEIILALVLGALKRL | 0.779 | −1394.13 | 0.000 | 0.000 |
| (SEQ ID NO: 61) | | | | | |

The ddG_ins and dscore values for each variant in this Table and Table 1 (except the manually added ones) fit the aforementioned criteria, indicating that the program works as intended. Finally, the program may combine the top three individual candidate mutations with respect to ddG_ins, and if the triple mutant passes the defined score threshold, it is also reported as a degreaser variant.
Table 2. A curated list of I53-dn5 pentamer degreaser variants that were experimentally characterized. The ddG_ins and dscore values for each variant in this Table and Table 1 (except the manually added ones) fit the aforementioned criteria, indicating that the program works as intended. Finally, the program may combine the top three individual candidate mutations with respect to ddG_ins, and if the triple mutant passes the defined score threshold, it is also reported as a degreaser variant.

TABLE 3
Previously described or naturally occurring proteins

| Protein | Protein | |
|---|---|---|
| name | type | Expressed sequence (optional residues in parentheses) |
| I3-01 | Original | (M)KMEELFKKHKIVAVLRANSVEEAKKKALAVFLGGVHLIEITFTVPDADTVI |
| | nanoparticle | KELSFLKEMGAIIGAGTVTSVEQCRKAVESGAEFIVSPHLDEEISQFCKEKGVF |
| | protein | YMPGVMTPTELVKAMKLGHTILKLFPGEVVGPQFVKAMKGPFPNVKFVPTGGVN |
| | | LDNVCEWFKAGVLAVGVGSALVKGTPVEVAEKAKAFVEKIRGCTE (SEQ ID |
| | | NO: 1) |
| O43-38 | Original | (M)ERNKLARQIIDTCLEMTRLGLNQGTAGSVSVRYQDGMLITPTGIPYEKLTE |
| tetramer | nanoparticle | SHIVFIDGNGKHEEGKLPQSEWRFHMAAYKARPDANAVVHNHAVHCTAVSILNR |
| | protein | SIPAIHYMIAAAGGNSIPCAPAATFGTDELSMLVAVALLNRKATLLQHHGLIAC |
| | | EVNLEKALWLAHEVEVLAQLYLTTLAITDPVPVLSDEEIAVVLEKFKTFGLRIE |
| | | (SEQ ID NO: 2) |
| O43-38 | Original | (M)KMEELFKKHKIVAVLRANSVVLALAKALAVFLGGVHLIEITFTVPDADTVI |
| trimer | nanoparticle | KELSFLKEKGAIIGAGTVTSVEQCRKAVESGAEFIVSPHLDEEISQFCKEKGVF |
| | protein | YMPGVMTPTELVKAMKLGHTILKLFPGEWGPQFVKAMKGPFPNVKFVPTGGVN |
| | | LDNVCEWFKAGVLAVGVGSALVKGEPEEVREKAKAFVEKIRGCTE(LEHHHHHH) |
| | | (SEQ ID NO: 3) |
| I53_dn5A | Original | (MG)KYDGSKLRIGILHARWNAEIILALVLGALKRLQEFGVKRENIIIETVPGS |
| (I53_dn5 | nanoparticle | FELPYGSKLFVEKQKRLGKPLDAIIPIGVLIKGSTMHFEYICDSTTHQLMKLNF |
| pentamer) | protein | ELGIPVIFGVLTCLTDEQAEARAGLIEGKMHNHGEDWGAAAVEMATKFN (SEQ |
| | | ID NO: 4) |
| hMPV F | Wild-type | (MSWKVVIIFSLLITPQHGLK)ESYLEESCSTITEGYLSVLRTGWYTNVFTLEV |
| (isolate | protein | GDVENLTCADGPSLIKTELDLTKSALRELRTVSADQLAREEQIENPRQSRFVLG |
| Arg/2/02) | | AIALGVATAAAVTAGVAIAKTIRLESEVTAIKNALKKTNEAVSTLGNGVRVLAT |
| | | AVRELKDFVSKNLTRAINKNKCDIADLKMAVSFSQFNRRFLNVVRQFSDNAGIT |
| | | PAISLDLMTDAELARAVSNMPTSAGQIKLMLENRAMVRRKGFGILIGVYGSSVI |
| | | YMVQLPIFGVIDTPCWIVKAAPSCSEKKGNYACLLREDQGWYCQNAGSTVYYPN |
| | | EKDCETRGDHVFCDTAAGINVAEQSKECNINISTTNYPCKVSTGRHPISMVALS |
| | | PLGALVACYKGVSCSIGSNRVGIIKQLNKGCSYITNQDADTVTIDNTVYQLSKV |
| | | EGEQHVIKGRPVSSSFDPVKFPEDQFNVALDQVFESIENSQALVDQSNRILSSA |
| | | EKGNT(GFIIVIILTAVLGSTMILVSVFIIIKKTKKPTGAPPELSGV)(SEQ |
| | | ID NO: 5) |
| hMPV_F | Prefusion | (MSWKVVIIFSLLITPQHGLK)ESYLEESCSTITEGYLSVLRTGWYTNVFTLEV |
| (aka 115- | hMPV F | GDVENLTCADGPSLIKTELDLTKSALRELRTVSADQLAREEQIENPRRRRFVLG |
| BV) | antigen | AIALGVATAAAVTAGVAIAKTIRLESEVTAIKNALKKTNEAVSTLGNGVRVLAT |
| | (Battles | AVRELKDFVSKNLTRAINKNKCDIPDLKMAVSFSQFNRRFLNVVRQFSDNAGIT |
| | et al., | PAISLDLMTDAELARAVSNMPTSAGQIKLMLENRAMVRRKGFGILIGVYGSSVI |
| | Nat. | YMVQLPIFGVIDTPCWIVKAAPSCSEKKGNYACLLREDQGWYCQNAGSTVYYPN |
| | Comms. | EKDCETRGDHVFCDTAAGINVAEQSKECNINISTTNYPCKVSTGRHPISMVALS |
| | 2017) | PLGALVACYKGVSCSIGSNRVGIIKQLNKGCSYITNQDADTVTIDNTVYQLSKV |
| | | EGEQHVIKGRPVSSSFDPVKFPEDQFNVALDQVFESIENSQALVDQSNRILSSA |
| | | EKGNT(SGRENLYFQGGGGSGYIPEAPRDGQAYVRKDGEWVLLSTFLGGIEGRH |
| | | HHHHH) (SEQ ID NO: 6) |

TABLE 4
Degreased sequences

| Protein | Protein | |
|---|---|---|
| name | type | Expressed sequence (optional residues in parentheses) |
| I3-01 | Degreased | (METDTLLLWVLLLWVPGSTGDYKDEK)MEELFKKHKIVAVLRANSVEEAKKKALAVF |
| H35D | nanoparticle | LGGVDLIEITFTVPDADTVIKELSFLKEMGAIIGAGTVTSVEQCRKAVESGAEFIVSP |
| | protein | HLDEEISQFCKEKGVFYMPGVMTPTELVKAMKLGHTILKLFPGEVVGPQFVKAMKGPF |
| | | PNVKFVPTGGVNLDNVCEWFKAGVLAVGVGSALVKGTPVEVAEKAKAFVEKIRGCTE |
| | | (QKLISEEDLHHHHHH) (SEQ ID NO: 7) |
| I3-01 | Degreased | (METDTLLLWVLLLWVPGSTGDYKDEK)MEELFKKHKIVAVLRANSVEEAKKDALAVF |
| K25D | nanoparticle | LGGVHLIEITFTVPDADTVIKELSFLKEMGAIIGAGTVTSVEQCRKAVESGAEFIVSP |
| | protein | HLDEEISQFCKEKGVFYMPGVMTPTELVKAMKLGHTILKLFPGEVVGPQFVKAMKGPF |
| | | PNVKFVPTGGVNLDNVCEWFKAGVLAVGVGSALVKGTPVEVAEKAKAFVEKIRGCTE |
| | | (QKLISEEDLHHHHHH) (SEQ ID NO: 8) |
| I3-01 | Degreased | (METDTLLLWVLLLWVPGSTGDYKDEK)MEELFKKHKIVAVLRANSVEEAKKNALAVF |
| K25N | nanoparticle | LGGVHLIEITFTVPDADTVIKELSFLKEMGAIIGAGTVTSVEQCRKAVESGAEFIVSP |
| | protein | HLDEEISQFCKEKGVFYMPGVMTPTELVKAMKLGHTILKLFPGEVVGPQFVKAMKGPF |
| | | PNVKFVPTGGVNLDNVCEWFKAGVLAVGVGSALVKGTPVEVAEKAKAFVEKIRGCTE |
| | | (QKLISEEDLHHHHHH) (SEQ ID NO: 9) |
| I3-01 | Degreased | (METDTLLLWVLLLWVPGSTGDYKDEK)MEELFKKHKIVAVLRANSVEEAKKKALAVF |
| L171Q | nanoparticle | LGGVHLIEITFTVPDADTVIKELSFLKEMGAIIGAGTVTSVEQCRKAVESGAEFIVSP |
| | protein | HLDEEISQFCKEKGVFYMPGVMTPTELVKAMKLGHTILKLFPGEVVGPQFVKAMKGPF |
| | | PNVKFVPTGGVNLDNVCEWFKAGVQAVGVGSALVKGTPVEVAEKAKAFVEKIRGCTE |
| | | (QKLISEEDLHHHHHH) (SEQ ID NO: 10) |
| I3-01 | Degreased | (METDTLLLWVLLLWVPGSTGDYKDEK)MEELFKKHKIVAVLRANSVEEAKKKALAVF |
| L171Q/ | nanoparticle | LGGVHLIEITFTVPDADTVIKELSFLKEMGAIIGAGTVTSVEQCRKAVESGAEFIVSP |
| S177E/ | protein | HLDEEISQFCKEKGVFYMPGVMTPTELVKAMKLGHTILKLFPGEVVGPQFVKAMKGPF |
| V180N | | PNVKFVPTGGVNLDNVCEWFKAGVQAVGVGEALNKGTPVEVAEKAKAFVEKIRGCTE |
| | | (QKLISEEDLHHHHHH) (SEQ ID NO: 11) |
| I3-01 | Degreased | (METDTLLLWVLLLWVPGSTGDYKDEK)MEELFKKHKIVAVLRANSVEEAKKKALAVF |
| ‘secretion | nanoparticle | LGGVDLIEITFTVPDADTVIKELSFLKEMGAIIGAGTVTSVEQCRKAVESGAEFIVSP |
| mutations’ | protein | HLDEEISQFCKEKGVFYMPGVMTPTELVKAMKLGHTILKLFPGEVVGPQFVKAMKGPF |
| (H35D/ | | PNVKFVPTGGVNLDNVCEWFKAGVQAVGVGEALNKGTPVEVAEKAKAFVEKIRGCTE |
| L171Q/ | | (QKLISEEDLHHHHHH) (SEQ ID NO: 12) |
| S177E/ | | |
| V180N) | | |
| I3-01 | Modified, | (METDTLLLWVLLLWVPGSTGD)MEELFKEHKIVAVLRANSVEEAKKKALAVFLGGVH |
| ‘negative | non- | LIEITFTVPDADTVIKELSFLKEMGAIIGAGTVTSVEQAREAVESGAEFIVSPHLDEE |
| interior’ | degreased | ISQFAKEEGVFYMPGVMTPTELVKAMKLGHTILKLFPGEWGPQFVEAMKGPFPNVKF |
| | nanoparticle | VPTGGVNLCNVAEWFEAGVLAVGVGSALVEGTPVEVAEKAKAFVEKIEGATE(QKLIS |
| | protein | EEDLHHHHHH) (SEQ ID NO: 13) |
| I3-01 | Degreased | (METDTLLLWVLLLWVPGSTGD)MEELFKEHKIVAVLRANSVEEAKKKALAVFLGGVD |
| ‘negative | nanoparticle | LIEITFTVPDADTVIKELSFLKEMGAIIGAGTVTSVEQAREAVESGAEFIVSPHLDEE |
| interior | protein | ISQFAKEEGVFYMPGVMTPTELVKAMKLGHTILKLFPGEWGPQFVEAMKGPFPNVKF |
| with | | VPTGGVNLDNVAEWFEAGVQAVGVGEALNEGTPVEVAEKAKAFVEKIEGATE(QKLIS |
| secretion | | EEDLHHHHHH) (SEQ ID NO: 14) |
| mutations’ | | |
| I53_dn5A | Degreased | (METDTLLLWVLLLWVPGSTGDYKDE)KKYDGSKLRIGILHARENAEI1LALVLGALK |
| W16E | nanoparticle | RLQEFGVKRENIIIETVPGSFELPYGSKLFVEKQKRLGKPLDAIIPIGVLIKGSTMHF |
| | protein | EYICDSTTHQLMKLNFELGIPVIFGVLTCLTDEQAEARAGLIEGKMHNHGEDWGAAAV |
| | | EMATKFN(LEGSEQKLISEEDLHHHHHH) (SEQ ID NO: 15) |
| I53_dn5A | Degreased | (METDTLLLWVLLLWVPGSTGDYKDE)KKYDGSKLRIGILHARWNAEI1LALVLGANK |
| L29N | nanoparticle | RLQEFGVKRENIIIETVPGSFELPYGSKLFVEKQKRLGKPLDAIIPIGVLIKGSTMHF |
| | protein | EYICDSTTHQLMKLNFELGIPVIFGVLTCLTDEQAEARAGLIEGKMHNHGEDWGAAAV |
| | | EMATKFN(LEGSEQKLISEEDLHHHHHH) (SEQ ID NO: 16) |
| I53_dn5A | Degreased | (METDTLLLWVLLLWVPGSTGDYKDE)KKYDGSKLRIGILHARWNAEIILALVLGALK |
| ‘mutl01’ | nanoparticle | RLQEFGVKRENIIIETVPGSFELPYGSKLFVEKQKRLGKPLDAIIPIGVLIKGSTMHF |
| [T116N/ | protein | EYICDSTTHQLMKLNFELGIPVIFGVLNCDKDEQAEARAGLIEGKMHNHGEDWGAAAV |
| L118D/ | | EMATKFN(LEGSEQKLISEEDLHHHHHH) (SEQ ID NO: 17) |
| T119K] | | |
| I53_dn5A | Degreased | (METDTLLLWVLLLWVPGSTGDYKDE)KKYDGSKLRIGILHARWNAEIILALVLGALK |
| T116N | nanoparticle | RLQEFGVKRENIIIETVPGSFELPYGSKLFVEKQKRLGKPLDAIIPIGVLIKGSTMHF |
| | protein | EYICDSTTHQLMKLNFELGIPVIFGVLNCLTDEQAEARAGLIEGKMHNHGEDWGAAAV |
| | | EMATKFN(LEGSEQKLISEEDLHHHHHH) (SEQ ID NO: 18) |
| I53_dn5A | Degreased | (METDTLLLWVLLLWVPGSTGDYKDE)KKYDGSKLRIGILHARWNAEIILALVLGALK |
| L118D | nanoparticle | RLQEFGVKRENIIIETVPGSFELPYGSKLFVEKQKRLGKPLDAIIPIGVLIKGSTMHF |
| | protein | EYICDSTTHQLMKLNFELGIPVIFGVLTCDTDEQAEARAGLIEGKMHNHGEDWGAAAV |
| | | EMATKFN(LEGSEQKLISEEDLHHHHHH) (SEQ ID NO: 19) |
| I53_dn5A | Degreased | (METDTLLLWVLLLWVPGSTGDYKDE)KKYDGSKLRIGILHARWNAEIILALVLGALK |
| T119K | nanoparticle | RLQEFGVKRENIIIETVPGSFELPYGSKLFVEKQKRLGKPLDAIIPIGVLIKGSTMHF |
| | protein | EYICDSTTHQLMKLNFELGIPVIFGVLTCLKDEQAEARAGLIEGKMHNHGEDWGAAAV |
| | | EMATKFN(LEGSEQKLISEEDLHHHHHH) (SEQ ID NO: 20) |
| I53_dn5A.1 | Degreased | (METDTLLLWVLLLWVPGSTGDYKDEMG)KYDGSKLRIGILHARGNAEIILALVLGAL |
| | nanoparticle | KRLQEFGVKRENIIIETVPGSFELPYGSKLFVEKQKRLGKPLDAIIPIGVLIRGSTPH |
| | protein | FDYIADSTTHQLMKLNFELGIPVIFGVITADTDEQAEARAGLIEGKMHNHGEDWGAAA |
| | | VEMATKFN(LEGSEQKLISEEDLHHHHHH) (SEQ ID NO: 21) |
| I53_dn5A.1 | Degreased | (METDTLLLWVLLLWVPGSTGDYKDEMG)KYDGSKLRIGILHARENAEIILALVLGAL |
| W16E | nanoparticle | KRLQEFGVKRENIIIETVPGSFELPYGSKLFVEKQKRLGKPLDAIIPIGVLIRGSTPH |
| | protein | FDYIADSTTHQLMKLNFELGIPVIFGVITADTDEQAEARAGLIEGKMHNHGEDWGAAA |
| | | VEMATKFN(LEGSEQKLISEEDLHHHHHH) (SEQ ID NO: 22) |
| I53_dn5A.2 | Degreased | (METDTLLLWVLLLWVPGSTGDYKDEMG)KYDGSKLRIGILHARGNAEIILELVLGAL |
| | nanoparticle | KRLQEFGVKRENIIIETVPGSFELPYGSKLFVEKQKRLGKPLDAIIPIGVLIRGSTAH |
| | protein | FDYIADSTTHQLMKLNFELGIPVIFGVLTTESDEQAEERAGTKAGNHGEDWGAAAVEM |
| | | ATKFN(LEGSEQKLISEEDLHHHHHH) (SEQ ID NO: 23) |
| O43- | Degreased | (METDTLLLWVLLLWVPGSTGDYKDESGM)ERNKLARQIIDTCLEMTRLGLNQGTAGS |
| 38tet | nanoparticle | VSVRYQDGMLITPTGIPYEKLTESHIVFIDGNGKHEEGKLPQSEWRFHMAAYKARPDA |
| N29S | protein | NAWHNHAVHCTAVSILNRSIPAIHYMIAAAGGNSIPCAPAATFGTDELSMLVEVALL |
| A141E | | NRKATLLQHHGLIACEVNLEKALWLAHEVEVLAQLYLTTLAITDPVPVLSDEEIAWL |
| | | EKFKTFGLRIEE(GSQKLISEEDLHHHHHH) (SEQ ID NO: 24) |
| O43- | Degreased | (METDTLLLWVLLLWVPGSTGDYKDESGM)NRNELARQIIDTMKEMTRLGLNQGTAGS |
| 38tet | nanoparticle | VSVRYQDGMLITPIGIPYEKLTEDHIVFIDGNGKHEEGKLPQSEWRFHMAAYKARPDA |
| “CysK | protein | NAWHNHAVHSTAVSILNREIPAIHYMIAAAGGNSIPSAPAATFGTDELSMLVEVALL |
| Olr- | | DRKATLLQHHGLIAVETNLEKALWLAHEVEVLAQLYLTTLAITDPVPVLSDEEIKTVL |
| Resurflr” | | EKFKTFGLRIEE(GSQKLISEEDLGSGSGSHHHHHH) (SEQ ID NO: 25) |
| N29S | | |
| A141E | | |
| O43- | Degreased | (METDTLLLWVLLLWVPGSTGDYKDEK)MEELFKKHKIVAVLRANSDVLALAKALAVF |
| 38tri | nanoparticle | LGGVHLIEITFTVPDADTVIKELSFLKEKGAIIGAGTVTSVEQCRKAVESGAEFIVSP |
| V19D | protein | HLDEEISQFCKEKGVFYMPGVMTPTELVKAMKLGHTILKLFPGEVVGPQFVKAMKGPF |
| | | PNVKFVPTGGVNLDNVCEWFKAGVLAVGVGSALVKGEPEEVREKAKAFVEKIRGC(GS |
| | | EQKLISEEDLHHHHHH) (SEQ ID NO: 26) |
| O43- | Degreased | (METDTLLLWVLLLWVPGSTGDYKDEK)MEELFKKHKIVAVLRANSVVEALAKALAVF |
| 38tri | nanoparticle | LGGVHLIEITFTVPDADTVIKELSFLKEKGAIIGAGTVTSVEQCRKAVESGAEFIVSP |
| L21E | protein | HLDEEISQFCKEKGVFYMPGVMTPTELVKAMKLGHTILKLFPGEVVGPQFVKAMKGPF |
| | | PNVKFVPTGGVNLDNVCEWFKAGVLAVGVGSALVKGEPEEVREKAKAFVEKIRGC(GS |
| | | EQKLISEEDLHHHHHH) (SEQ ID NO: 27) |
| O43- | Degreased | (METDTLLLWVLLLWVPGSTGDYKDEK)MEELFKKHKIVAVLRANSVVLALAKALAVF |
| 38tri | nanoparticle | NGGVHLIEITFTVPDADTVIKELSFLKEKGAIIGAGTVTSVEQCRKAVESGAEFIVSP |
| L31N | protein | HLDEEISQFCKEKGVFYMPGVMTPTELVKAMKLGHTILKLFPGEVVGPQFVKAMKGPF |
| | | PNVKFVPTGGVNLDNVCEWFKAGVLAVGVGSALVKGEPEEVREKAKAFVEKIRGC(GS |
| | | EQKLISEEDLHHHHHH) (SEQ ID NO: 28) |
| hMPV_F- | Degreased | (MSWKWIIFSLLITPQHG)LKESYLEESCSTITEGYLSVLRTGWYTNVFTLEVGDVE |
| 50A_13 | hMPV | NLTCADGPSLIKTELDLTKSALRELRTVSADQLAREEQIENPRRRRFVLGAIALGVAE |
| | F | AAARTAGVAIAKTIRLESEVTAIKNALKKTNEAVSTLGNGVRVLATAVRELKDFVSKN |
| | protein | LTRAINKNKCDIPDLKMAVSFSQFNRRFLNVVRQFSDNAGITPAISLDLMTDAELARA |
| | T114E + | VSNMPTSAGQIKLMLENRAMVRRKGFGILIGVYGSSVIYMVQLPIFGVIDTPCWIVKA |
| | V118R | APSCSEKKGNYACLLREDQGWYCQNAGSTVYYPNEKDCETRGDHVFCDTAAGINVAEQ |
| | | SKECNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQFNVALDQ |
| | | VFESIENSQALVDQSNRILSSAEKGNT (SEQ ID NO: 29) |
| | | (MSWKWIIFSLLITPQHG)LKESYLEESCSTITEGYLSVLRTGWYTNVFTLEVGDVE |
| | | NLTCADGPSLIKTELDLTKSALRELRTVSADQLAREEQIENPRRRRFVLGAIALGVAE |
| | | AAARTAGVAIAKTIRLESEVTAIKNALKKTNEAVSTLGNGVRVLATAVRELKDFVSKN |
| | | LTRAINKNKCDIPDLKMAVSFSQFNRRFLNWRQFSDNAGITPAISLDLMTDAELARA |
| | | VSNMPTSAGQIKLMLENRAMVRRKGFGILIGVYGSSVIYMVQLPIFGVIDTPCWIVKA |
| | | APSCSEKKGNYACLLREDQGWYCQNAGSTVYYPNEKDCETRGDHVFCDTAAGINVAEQ |
| | | SKECNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQFNVALDQ |
| | | VFESIENSQALVDQSNRILSSAEKGNT(SGRENLYFQGGGGSGYIPEAPRDGQAYVRK |
| | | DGEWVLLSTFLGSGGSGSGSGGSGSGEKAAKAEEAAR)KMEELFKKHKIVAVLRANSV |
| | | EEAIEKAVAVFAGGVHLIEITFTVPDADTVIKALSVLKEKGAIIGAGTVTSVEQCRKA |
| | | VESGAEFIVSPHLDEEISQFCKEKGVFYMPGVMTPTELVKAMKLGHTILKLFPGEVVG |
| | | PQFVKAMKGPFPNVKFVPTGGVNLDNVCEWFKAGVLAVGVGSALVKGTPDEVREKAKA |
| | | FVEKIRGCTE(LEHHHHHH) (SEQ ID NO: 30) |
| hMPV_F- | Degreased | (MSWKWIIFSLLITPQHG)LKESYLEESCSTITEGYLSVLRTGWYTNVFTLEVGDVE |
| 50A_14 | hMPV | NLTCADGPSLIKTELDLTKSALRELRTVSADQLAREEQIENPRRRRFVLGDIALGRAE |
| | F | AAARTAGVAIAKTIRLESEVTAIKNALKKTNEAVSTLGNGVRVLATAVRELKDFVSKN |
| | protein | LTRAINKNKCDIPDLKMAVSFSQFNRRFLNVVRQFSDNAGITPAISLDLMTDAELARA |
| | A107D + | VSNMPTSAGQIKLMLENRAMVRRKGFGILIGVYGSSVIYMVQLPIFGVIDTPCWIVKA |
| | V112R + | APSCSEKKGNYACLLREDQGWYCQNAGSTVYYPNEKDCETRGDHVFCDTAAGINVAEQ |
| | T114E + | SKECNINISTTNYPCKVSTGRHPISMVALSPLGALVACYKGVSCSIGSNRVGIIKQLN |
| | V118R | KGCSYITNQDADTVTIDNTVYQLSKVEGEQHVIKGRPVSSSFDPVKFPEDQFNVALDQ |
| | | VFESIENSQALVDQSNRILSSAEKGNT (SEQ ID NO: 31) |
| | | (MSWKWIIFSLLITPQHG)LKESYLEESCSTITEGYLSVLRTGWYTNVFTLEVGDVE |
| | | NLTCADGPSLIKTELDLTKSALRELRTVSADQLAREEQIENPRRRRFVLGDIALGRAE |
| | | AAARTAGVAIAKTIRLESEVTAIKNALKKTNEAVSTLGNGVRVLATAVRELKDFVSKN |
| | | LTRAINKNKCDIPDLKMAVSFSQFNRRFLNWRQFSDNAGITPAISLDLMTDAELARA |
| | | VSNMPTSAGQIKLMLENRAMVRRKGFGILIGVYGSSVIYMVQLPIFGVIDTPCWIVKA |
| | | APSCSEKKGNYACLLREDQGWYCQNAGSTVYYPNEKDCETRGDHVFCDTAAGINVAEQ |
| | | SKECNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQFNVALDQ |
| | | VFESIENSQALVDQSNRILSSAEKGNT(SGRENLYFQGGGGSGYIPEAPRDGQAYVRK |
| | | DGEWVLLSTFLGSGGSGSGSGGSGSGEKAAKAEEAAR)KMEELFKKHKIVAVLRANSV |
| | | EEAIEKAVAVFAGGVHLIEITFTVPDADTVIKALSVLKEKGAIIGAGTVTSVEQCRKA |
| | | VESGAEFIVSPHLDEEISQFCKEKGVFYMPGVMTPTELVKAMKLGHTILKLFPGEVVG |
| | | PQFVKAMKGPFPNVKFVPTGGVNLDNVCEWFKAGVLAVGVGSALVKGTPDEVREKAKA |
| | | FVEKIRGCTE(LEHHHHHH) (SEQ ID NO: 32) |
| hMPV_F- | Degreased | (MSWKWIIFSLLITPQHG)LKESYLEESCSTITEGYLSVLRTGWYTNVFTLEVGDVE |
| 50A_15 | hMPV | NLTCADGPSLIKTELDLTKSALRELRTVSADQLAREEQIENPRRRRFVLGDIALGRAT |
| | F | AAAVTAGVAIAKTIRLESEVTAIKNALKKTNEAVSTLGNGVRVLATAVRELKDFVSKN |
| | protein | LTRAINKNKCDIPDLKMAVSFSQFNRRFLNWRQFSDNAGITPAISLDLMTDAELARA |
| | A107D + | VSNMPTSAGQIKLMLENRAMVRRKGFGILIGVYGSSVIYMVQLPIFGVIDTPCWIVKA |
| | V112R | APSCSEKKGNYACLLREDQGWYCQNAGSTVYYPNEKDCETRGDHVFCDTAAGINVAEQ |
| | | SKECNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQFNVALDQ |
| | | VFESIENSQALVDQSNRILSSAEKGNT (SEQ ID NO: 33) |
| | | (MSWKWIIFSLLITPQHG)LKESYLEESCSTITEGYLSVLRTGWYTNVFTLEVGDVE |
| | | NLTCADGPSLIKTELDLTKSALRELRTVSADQLAREEQIENPRRRRFVLGDIALGRAT |
| | | AAAVTAGVAIAKTIRLESEVTAIKNALKKTNEAVSTLGNGVRVLATAVRELKDFVSKN |
| | | LTRAINKNKCDIPDLKMAVSFSQFNRRFLNWRQFSDNAGITPAISLDLMTDAELARA |
| | | VSNMPTSAGQIKLMLENRAMVRRKGFGILIGVYGSSVIYMVQLPIFGVIDTPCWIVKA |
| | | APSCSEKKGNYACLLREDQGWYCQNAGSTVYYPNEKDCETRGDHVFCDTAAGINVAEQ |
| | | SKECNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQFNVALDQ |
| | | VFESIENSQALVDQSNRILSSAEKGNT(SGRENLYFQGGGGSGYIPEAPRDGQAYVRK |
| | | DGEWVLLSTFLGSGGSGSGSGGSGSGEKAAKAEEAAR)KMEELFKKHKIVAVLRANSV |
| | | EEAIEKAVAVFAGGVHLIEITFTVPDADTVIKALSVLKEKGAIIGAGTVTSVEQCRKA |
| | | VESGAEFIVSPHLDEEISQFCKEKGVFYMPGVMTPTELVKAMKLGHTILKLFPGEVVG |
| | | PQFVKAMKGPFPNVKFVPTGGVNLDNVCEWFKAGVLAVGVGSALVKGTPDEVREKAKA |
| | | FVEKIRGCTE(LEHHHHHH) (SEQ ID NO: 34) |
| hMPV_F_dn5 | Degreased | (MSWKWIIFSLLITPQHG)LKESYLEESCSTITEGYLSVLRTGWYTNVFTLEVGDVE |
| B_01 | hMPV | NLTCADGPSLIKTELDLTKSALRELRTVSADQLAREEQIENPRRRRFVLGDIALGRAE |
| | F protein | AAARTAGVAIAKTIRLESEVTAIKNALKKTNEAVSTLGNGVRVLATAVRELKDFVSKN |
| | A107D + | LTRAINKNKCDIPDLKMAVSFSQFNRRFLNWRQFSDNAGITPAISLDLMTDAELARA |
| | V112R + | VSNMPTSAGQIKLMLENRAMVRRKGFGILIGVYGSSVIYMVQLPIFGVIDTPCWIVKA |
| | T114E + | APSCSEKKGNYACLLREDQGWYCQNAGSTVYYPNEKDCETRGDHVFCDTYAGINVAEQ |
| | V118R | SKECNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQFNVALDQ |
| | | VFESIENSQALVDQSNRILSSAEKGNT (SEQ ID NO: 35) |
| | | (MSWKWIIFSLLITPQHG)LKESYLEESCSTITEGYLSVLRTGWYTNVFTLEVGDVE |
| | | NLTCADGPSLIKTELDLTKSALRELRTVSADQLAREEQIENPRRRRFVLGDIALGRAE |
| | | AAARTAGVAIAKTIRLESEVTAIKNALKKTNEAVSTLGNGVRVLATAVRELKDFVSKN |
| | | LTRAINKNKCDIPDLKMAVSFSQFNRRFLNVVRQFSDNAGITPAISLDLMTDAELARA |
| | | VSNMPTSAGQIKLMLENRAMVRRKGFGILIGVYGSSVIYMVQLPIFGVIDTPCWIVKA |
| | | APSCSEKKGNYACLLREDQGWYCQNAGSTVYYPNEKDCETRGDHVFCDTAAGINVAEQ |
| | | SKECNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQFNVALDQ |
| | | VFESIENSQALVDQSNRILSSAEKGNT(SGRENLYFQGSG)EEAELAYLLGELAYKLG |
| | | EYRIAIRAYRIALKRDPNNAEAWYNLGNAYYKQGRYREAIEYYQKALELDPNNAEAWY |
| | | NLGNAYYERGEYEEAIEYYRKALRLDPNNADAMQNLLNAKMREE(GGWELQHHHHHH) |
| | | (SEQ ID NO: 36) |
| hMPV_F_dn5 | Degreased | (MSWKWIIFSLLITPQHG)LKESYLEESCSTITEGYLSVLRTGWYTNVFTLEVGDVE |
| B_02 | hMPV | NLTCADGPSLIKTELDLTKSALRELRTVSADQLAREEQIENPRRRRFVLGDIALGRAE |
| | F | AAARTAGVAIAKTIRLESEVTAIKNALKKTNEAVSTLGNGVRVLATAVRELKDFVSKN |
| | protein | LTRAINKNKCDIPDLKMAVSFSQFNRRFLNWRQFSDNAGITPAISLDLMTDAELARA |
| | A107D + | VSNMPTSAGQIKLMLENRAMVRRKGFGILIGVYGSSVIYMVQLPIFGVIDTPCWIVKA |
| | V112R + | APSCSEKKGNYACLLREDQGWYCQNAGSTVYYPNEKDCETRGDHVFCDTAAGINVAEQ |
| | T114E + | SKECNINISTTNYPCKVSTGRHPISMVALSPLGALVACYKGVSCSIGSNRVGIIKQLN |
| | V118R | KGCSYITNQDADTVTIDNTVYQLSKVEGEQHVIKGRPVSSSFDPVKFPEDQFNVALDQ |
| | | VFESIENSQALVDQSNRILSSAEKGNT (SEQ ID NO: 37) |
| | | (MSWKWIIFSLLITPQHG)LKESYLEESCSTITEGYLSVLRTGWYTNVFTLEVGDVE |
| | | NLTCADGPSLIKTELDLTKSALRELRTVSADQLAREEQIENPRRRRFVLGDIALGRAE |
| | | AAARTAGVAIAKTIRLESEVTAIKNALKKTNEAVSTLGNGVRVLATAVRELKDFVSKN |
| | | LTRAINKNKCDIPDLKMAVSFSQFNRRFLNWRQFSDNAGITPAISLDLMTDAELARA |
| | | VSNMPTSAGQIKLMLENRAMVRRKGFGILIGVYGSSVIYMVQLPIFGVIDTPCWIVKA |
| | | APSCSEKKGNYACLLREDQGWYCQNAGSTVYYPNEKDCETRGDHVFCDTAAGINVAEQ |
| | | SKECNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQFNVALDQ |
| | | VFESIENSQALVDQSNRILSSAEKGNT(SGRENLYFQGSGS)EEAELAYLLGELAYKL |
| | | GEYRIAIRAYRIALKRDPNNAEAWYNLGNAYYKQGRYREAIEYYQKALELDPNNAEAW |
| | | YNLGNAYYERGEYEEAIEYYRKALRLDPNNADAMQNLLNAKMREE(GGWELQHHHHHH) |
| | | (SEQ ID NO: 38) |
| hMPV_F_dn5 | Degreased | (MSWKWIIFSLLITPQHG)LKESYLEESCSTITEGYLSVLRTGWYTNVFTLEVGDVE |
| B_03 | hMPV | NLTCADGPSLIKTELDLTKSALRELRTVSADQLAREEQIENPRRRRFVLGDIALGRAE |
| | F | AAARTAGVAIAKTIRLESEVTAIKNALKKTNEAVSTLGNGVRVLATAVRELKDFVSKN |
| | protein | LTRAINKNKCDIPDLKMAVSFSQFNRRFLNWRQFSDNAGITPAISLDLMTDAELARA |
| | A107D + | VSNMPTSAGQIKLMLENRAMVRRKGFGILIGVYGSSVIYMVQLPIFGVIDTPCWIVKA |
| | V112R + | APSCSEKKGNYACLLREDQGWYCQNAGSTVYYPNEKDCETRGDHVFCDTAAGINVAEQ |
| | T114E + | SKECNINISTTNYPCKVSTGRHPISMVALSPLGALVACYKGVSCSIGSNRVGIIKQLN |
| | V118R | KGCSYITNQDADTVTIDNTVYQLSKVEGEQHVIKGRPVSSSFDPVKFPEDQFNVALDQ |
| | | VFESIENSQALVDQSNRILSSAEKGNT (SEQ ID NO: 39) |
| | | (MSWKWIIFSLLITPQHG)LKESYLEESCSTITEGYLSVLRTGWYTNVFTLEVGDVE |
| | | NLTCADGPSLIKTELDLTKSALRELRTVSADQLAREEQIENPRRRRFVLGDIALGRAE |
| | | AAARTAGVAIAKTIRLESEVTAIKNALKKTNEAVSTLGNGVRVLATAVRELKDFVSKN |
| | | LTRAINKNKCDIPDLKMAVSFSQFNRRFLNWRQFSDNAGITPAISLDLMTDAELARA |
| | | VSNMPTSAGQIKLMLENRAMVRRKGFGILIGVYGSSVIYMVQLPIFGVIDTPCWIVKA |
| | | APSCSEKKGNYACLLREDQGWYCQNAGSTVYYPNEKDCETRGDHVFCDTAAGINVAEQ |
| | | SKECNINISTTNYPCKVSTGRHPISMVALSPLGALVACYKGVSCSIGSNRVGIIKQLN |
| | | KGCSYITNQDADTVTIDNTVYQLSKVEGEQHVIKGRPVSSSFDPVKFPEDQFNVALDQ |
| | | VFESIENSQALVDQSNRILSSAEKGNT(SGRENLYFQGSGSG)EEAELAYLLGELAYK |
| | | LGEYRIAIRAYRIALKRDPNNAEAWYNLGNAYYKQGRYREAIEYYQKALELDPNNAEA |
| | | WYNLGNAYYERGEYEEAIEYYRKALRLDPNNAD7XMQNLLNAKMREE(GGWELQHHHHH |
| | | H) (SEQ ID NO: 40) |
| hMPV_F_dn5 | Degreased | (MSWKWIIFSLLITPQHG)LKESYLEESCSTITEGYLSVLRTGWYTNVFTLEVGDVE |
| B_04 | hMPV | NLTCADGPSLIKTELDLTKSALRELRTVSADQLAREEQIENPRRRRFVLGDIALGRAE |
| | F | AAARTAGVAIAKTIRLESEVTAIKNALKKTNEAVSTLGNGVRVLATAVRELKDFVSKN |
| | protein | LTRAINKNKCDIPDLKMAVSFSQFNRRFLNVVRQFSDNAGITPAISLDLMTDAELARA |
| | A107D + | VSNMPTSAGQIKLMLENRAMVRRKGFGILIGVYGDSSVIYMVQLPIFGVIDTPCWIVK |
| | V112R + | AAPSCSEKKGNYACLLREDQGWYCQNAGSTVYYPNEKDCETRGDHVFCDTAAGINVAE |
| | T114E + | QSKECNINISTTNYPCKVSTGRHPISMVALSPLGALVACYKGVSCSIGSNRVGIIKQL |
| | V118R + | NKGCSYITNQDADTVTIDNTVYQLSKVEGEQHVIKGRPVSSSFDPVKFPEDQFNVALD |
| | G264D | QVFESIENSQALVDQSNRILSSAEKGNT (SEQ ID NO: 41) |
| | | (MSWKWIIFSLLITPQHG)LKESYLEESCSTITEGYLSVLRTGWYTNVFTLEVGDVE |
| | | NLTCADGPSLIKTELDLTKSALRELRTVSADQLAREEQIENPRRRRFVLGDIALGRAE |
| | | AAARTAGVAIAKTIRLESEVTAIKNALKKTNEAVSTLGNGVRVLATAVRELKDFVSKN |
| | | LTRAINKNKCDIPDLKMAVSFSQFNRRFLNWRQFSDNAGITPAISLDLMTDAELARA |
| | | VSNMPTSAGQIKLMLENRAMVRRKGFGILIGVYGDSSVIYMVQLPIFGVIDTPCWIVK |
| | | AAPSCSEKKGNYACLLREDQGWYCQNAGSTVYYPNEKDCETRGDHVFCDTAAGINVAE |
| | | QSKECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQFNVALD |
| | | QVFESIENSQALVDQSNRILSSAEKGNT(SGRENLYFQGSG)EEAELAYLLGELAYKL |
| | | GEYRIAIRAYRIALKRDPNNAEAWYNLGNAYYKQGRYREAIEYYQKALELDPNNAEAW |
| | | YNLGNAYYERGEYEEAIEYYRKALRLDPNNADAMQNLLNAKMREE(GGWELQHHHHHH) |
| | | (SEQ ID NO: 42) |
| hMPV_F_dn5 | Degreased | (MSWKWIIFSLLITPQHG)LKESYLEESCSTITEGYLSVLRTGWYTNVFTLEVGDVE |
| B_05 | hMPV | NLTCADGPSLIKTELDLTKSALRELRTVSADQLAREEQIENPRRRRFVLGDIALGRAE |
| | F | AAARTAGVAIAKTIRLESEVTAIKNALKKTNEAVSTLGNGVRVLATAVRELKDFVSKN |
| | protein | LTRAINKNKCDIPDLKMAVSFSQFNRRFLNWRQFSDNAGITPAISLDLMTDAELARA |
| | A107D + | VSNMPTSAGQIKLMLENRAMVRRKGFGILIGVYGDSSVIYMVQLPIFGVIDTPCWIVK |
| | V112R + | AAPSCSEKKGNYACLLREDQGWYCQNAGSTVYYPNEKDCETRGDHVFCDTAAGINVAE |
| | T114E + | QSKECNINISTTNYPCKVSTGRHPISMVALSPLGALVACYKGVSCSIGSNRVGIIKQL |
| | V118R + | NKGCSYITNQDADTVTIDNTVYQLSKVEGEQHVIKGRPVSSSFDPVKFPEDQFNVALD |
| | G264D | QVFESIENSQALVDQSNRILSSAEKGNT (SEQ ID NO: 43) |
| | | (MSWKWIIFSLLITPQHG)LKESYLEESCSTITEGYLSVLRTGWYTNVFTLEVGDVE |
| | | NLTCADGPSLIKTELDLTKSALRELRTVSADQLAREEQIENPRRRRFVLGDIALGRAE |
| | | AAARTAGVAIAKTIRLESEVTAIKNALKKTNEAVSTLGNGVRVLATAVRELKDFVSKN |
| | | LTRAINKNKCDIPDLKMAVSFSQFNRRFLNWRQFSDNAGITPAISLDLMTDAELARA |
| | | VSNMPTSAGQIKLMLENRAMVRRKGFGILIGVYGDSSVIYMVQLPIFGVIDTPCWIVK |
| | | AAPSCSEKKGNYACLLREDQGWYCQNAGSTVYYPNEKDCETRGDHVFCDTAAGINVAE |
| | | QSKECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQFNVALD |
| | | QVFESIENSQALVDQSNRILSSAEKGNT(SGRENLYFQGSGS)EEAELAYLLGELAYK |
| | | LGEYRIAIRAYRIALKRDPNNAEAWYNLGNAYYKQGRYREAIEYYQKALELDPNNAEA |
| | | WYNLGNAYYERGEYEEAIEYYRKALRLDPNNADAMQNLLNAKMREE(GGWELQHHHHH |
| | | H) (SEQ ID NO: 44) |
| hMPV_F_dn5 | Degreas | (MSWKWIIFSLLITPQHG)LKESYLEESCSTITEGYLSVLRTGWYTNVFTLEVGDVE |
| B_06 | ed hMPV | NLTCADGPSLIKTELDLTKSALRELRTVSADQLAREEQIENPRRRRFVLGDIALGRAE |
| | F | AAARTAGVAIAKTIRLESEVTAIKNALKKTNEAVSTLGNGVRVLATAVRELKDFVSKN |
| | protein | LTRAINKNKCDIPDLKMAVSFSQFNRRFLNWRQFSDNAGITPAISLDLMTDAELARA |
| | A107D + | VSNMPTSAGQIKLMLENRAMVRRKGFGILIGVYGDSSVIYMVQLPIFGVIDTPCWIVK |
| | V112R + | AAPSCSEKKGNYACLLREDQGWYCQNAGSTVYYPNEKDCETRGDHVFCDTAAGINVAE |
| | T114E + | QSKECNINISTTNYPCKVSTGRHPISMVALSPLGALVACYKGVSCSIGSNRVGIIKQL |
| | V118R + | NKGCSYITNQDADTVTIDNTVYQLSKVEGEQHVIKGRPVSSSFDPVKFPEDQFNVALD |
| | G264D | QVFESIENSQALVDQSNRILSSAEKGNT (SEQ ID NO: 45) |
| | | (MSWKWIIFSLLITPQHG)LKESYLEESCSTITEGYLSVLRTGWYTNVFTLEVGDVE |
| | | NLTCADGPSLIKTELDLTKSALRELRTVSADQLAREEQIENPRRRRFVLGDIALGRAE |
| | | AAARTAGVAIAKTIRLESEVTAIKNALKKTNEAVSTLGNGVRVLATAVRELKDFVSKN |
| | | LTRAINKNKCDIPDLKMAVSFSQFNRRFLNVVRQFSDNAGITPAISLDLMTDAELARA |
| | | VSNMPTSAGQIKLMLENRAMVRRKGFGILIGVYGDSSVIYMVQLPIFGVIDTPCWIVK |
| | | AAPSCSEKKGNYACLLREDQGWYCQNAGSTVYYPNEKDCETRGDHVFCDTAAGINVAE |
| | | QSKECNINISTTNYPCKVSTGRHPISMVALSPLGALVACYKGVSCSIGSNRVGIIKQL |
| | | NKGCSYITNQDADTVTIDNTVYQLSKVEGEQHVIKGRPVSSSFDPVKFPEDQFNVALD |
| | | QVFESIENSQALVDQSNRILSSAEKGNT(SGRENLYFQGSGSG)EEAELAYLLGELAY |
| | | KLGEYRIAIRAYRIALKRDPNNAEAWYNLGNAYYKQGRYREAIEYYQKALELDPNNAE |
| | | AWYNLGNAYYERGEYEEAIEYYRKALRLDPNNADAMQNLLNAKMREE(GGWELQHHHH |
| | | HH) (SEQ ID NO: 46) |

From the foregoing, it will be appreciated that, although specific embodiments of the disclosure have been described herein for purposes of illustration, various modifications may be made without deviating from the spirit and scope of the invention. Accordingly, the invention is not limited except as by the appended claims.

We claim:

1. A polypeptide comprising or consisting of:
(a) an amino acid sequence at least 75%, 80%, 85%, 90%, 91%, 92%, 93%, 94%, 95%, 96%, 97%, 98%, or 99% identical to the amino acid sequence of SEQ ID NO:1 (I3-01 wild type), wherein 1, 2, 3, 4, 5, 6, 7, 8, 9, or all 10 of the following mutations relative to SEQ ID NO:1 are present in the polypeptide: F32Y, H37D/E/K/N/Q/R, F43Q, F168D/E/K/N/Q/R/S/T/Y, K169D/E/N/Q, L173D/E/N/Q/S, A174S, S179D/E, K183D/E, and/or T185D/E/K/N/Q/S;
(b) an amino acid sequence at least 75%, 80%, 85%, 90%, 91%, 92%, 93%, 94%, 95%, 96%, 97%, 98%, or 99% identical to the amino acid sequence of SEQ ID NO:2 (O43-38 tetramer wild type), wherein 1, 2, 3, 4, 5, 6, 7, 8, or all 9 of the following mutations relative to SEQ ID NO:2 are present in the polypeptide: M138D/E/K/N/Q/R/S/T, L139D/N/S, A141S, V142R/T, A143S, N146D/E/K/R, R147N, H172D/E/K/N/Q, and/or E173D/K.
(c) an amino acid sequence at least 75%, 80%, 85%, 90%, 91%, 92%, 93%, 94%, 95%, 96%, 97%, 98%, or 99% identical to the amino acid sequence of SEQ ID NO:3 (O43-38 trimer wild type), wherein 1, 2, 3, 4, 5, 6, 7, 8, 9, 10, 11, 12, 13, 14, 15, 16, 17, 18, 19, 20, or all 21 of the following mutations relative to SEQ ID NO:3 are present in the polypeptide: R17D/E/K/N/Q/S/T, N19D/E, S20D/E/K/N, V21D/T, V22D/E/Q/S/T, L23D/E/K/N/Q/R/S, A26S, K27N/Q, A30S V31N/S/T, F32R/Y, L33D/E/K/N/Q/R/S/T, H37D/E/K/N/R, F43Q, W167D/E/K/N/Q/R/S/T/Y, F168D/E/K/N/Q/R/S/T/Y, K169D/E/N, L173D/E/N/Q/R/S, A174S, S179D/E/K/N/Q/R, and/or K183D/E/N/Q;
(d) an amino acid sequence at least 75%, 80%, 85%, 90%, 91%, 92%, 93%, 94%, 95%, 96%, 97%, 98%, or 99% identical to the amino acid sequence of SEQ ID NO:4 (I53_dn5A wild type), wherein 1, 2, 3, 4, 5, 6, 7, 8, 9, or all 10 of the following mutations relative to SEQ ID NO:4 are present in the polypeptide: R17T, W18D/E/K/N/Q/R/S/T/Y, N19E, E21D, L28D/E/K/N/Q/R/S/T/Y, L31D/E/K/N/Q/S/T, K32D/E/N/Q, T118D/E/N/Q/S, L120D/E/K/N/Q/R/S/T, and/or T121D/E/K/N/S; or
(e) an amino acid sequence at least 75%, 80%, 85%, 90%, 91%, 92%, 93%, 94%, 95%, 96%, 97%, 98%, or 99% identical to the amino acid sequence of SEQ ID NO:5 or 6 (hMPV wild type), wherein 1, 2, 3, 4, or all 5, of the following mutations relative to SEQ ID NO:5 or 6 are present in the polypeptide: A107D, V112R, T114E, V118R, and/or G264D;
wherein residues in parentheses are optional and may be present or may be absent in whole or in part.

2. The polypeptide of claim 1, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:1 (I3-01 wild type), wherein 1, 2, 3, 4, 5, 6, 7, 8, 9, or all 10 of the following mutations relative to SEQ ID NO:1 are present in the polypeptide: F32Y, H37D/E/K/N/Q/R, F43Q, F168D/E/K/N/Q/R/S/T/Y, K169D/E/N/Q, L173D/E/N/Q/S, A174S, S179D/E, K183D/E, and/or T185D/E/K/N/Q/S.

3. The polypeptide of claim 2, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:7-14.

4. The polypeptide of claim 1, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:2 (O43-38 tetramer wild type), wherein 1, 2, 3, 4, 5, 6, 7, 8, or all 9 of the following mutations relative to SEQ ID NO:2 are present in the polypeptide: M138D/E/K/N/Q/R/S/T, L139D/N/S, A141S, V142R/T, A143S, N146D/E/K/R, R147N, H172D/E/K/N/Q, and/or E173D/K.

5. The polypeptide of claim 4, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:24-25.

6. The polypeptide of claim 1, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:3 (O43-38 trimer wild type), wherein 1, 2, 3, 4, 5, 6, 7, 8, 9, 10, 11, 12, 13, 14, 15, 16, 17, 18, 19, 20, or all 21 of the following mutations relative to SEQ ID NO:3 are present in the polypeptide: R17D/E/K/N/Q/S/T, N19D/E, S20D/E/K/N, V21D/T, V22D/E/Q/S/T, L23D/E/K/N/Q/R/S, A26S, K27N/Q, A30S V31N/S/T, F32R/Y, L33D/E/K/N/Q/R/S/T, H37D/E/K/N/R, F43Q, W167D/E/K/N/Q/R/S/T/Y, F168D/E/K/N/Q/R/S/T/Y, K169D/E/N, L173D/E/N/Q/R/S, A174S, S179D/E/K/N/Q/R, and/or K183D/E/N/Q.

7. The polypeptide of claim 6, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:26-28.

8. The polypeptide of claim 1, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:4 (I53_dn5A), wherein 1, 2, 3, 4, 5, 6, 7, 8, 9, or all 10 of the following mutations relative to SEQ ID NO:4 are present in the polypeptide: R17T, W18D/E/K/N/Q/R/S/T/Y, N19E, E21D, L28D/E/K/N/Q/R/S/T/Y, L31D/E/K/N/Q/S/T, K32D/E/N/Q, T118D/E/N/Q/S, L120D/E/K/N/Q/R/S/T, and/or T121D/E/K/N/S.

9. The polypeptide of claim 8, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:15-23.

10. The polypeptide of claim 1, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:5 (hMPV wild type) or 6 (hMPV or 115-BV), wherein 1, 2, 3, 4, or all 5, of the following mutations relative to SEQ ID NO:5 or 6 are present in the polypeptide: A107D, V112R, T114E, V118R, and/or G264D.

11. The polypeptide of claim 8, wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:5 or 6, wherein the polypeptide comprises a set of mutations relative to SEQ ID NO:5 or 6 selected from the group consisting of:
(a) T114E+V118R
(b) A107D+V112R+T114E+V118R
(c) A107D+V112R
(d) A107D+V112R+T114E+V118R; and
(e) A107D+V112R+T114E+V118R+G264D; or
wherein the polypeptide comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:29, 31, 33, 35, 37, 39, 41, 43, or 45, wherein residues in parentheses are optional and may be present or may be absent in whole or in part.

12. The polypeptide of any one of claims 1-11, wherein some or all of the residues in parentheses are absent.

13. The polypeptide of any one of claims 1-11, wherein some or all of the residues in parentheses are present.

14. A fusion protein comprising:
(a) the polypeptide according to any one of claims 2-9; and
(b) a second functional polypeptide.

15. The fusion protein of claim 14, wherein the second functional polypeptide comprises an immunogenic portion of a polypeptide antigen.

16. The fusion protein of claim 14 or 15, wherein the second functional polypeptide comprises the polypeptide of claim 10 or 11, or wherein the fusion protein comprises or consists of an amino acid sequence at least 75%, 80%, 85%, 90%, 91%, 92%, 93%, 94%, 95%, 96%, 97%, 98%, or 99% identical to the amino acid sequence of SEQ ID NO:30, 32, 34, 36, 38, 40, 42, 44, or 46 wherein residues in parentheses are optional and may be present or may be absent in whole or in part.

17. A nanoparticle comprising a plurality of the polypeptides or fusion proteins of any one of claims 1-16.

18. The nanoparticle of claim 17, wherein the nanoparticle comprises
(a) a plurality of polypeptides according to claim 2 or 3, or
(b) a plurality of fusion proteins comprising a plurality of polypeptides according to claim 2 or 3, wherein one or more of the fusion proteins comprise a second functional polypeptide, including but not limited to an immunogenic portion of a polypeptide antigen, wherein the polypeptide antigen includes but is not limited to the polypeptide of claim 10 or 11.

19. The nanoparticle of claim 17, wherein the nanoparticle comprises
(a) a plurality of first polypeptides according to claim 4 or 5 that self-interact to form a first multimeric substructure; and
(b) a plurality of second polypeptides according to claim 6 or 7 that self-interact to form a second multimeric substructure;
wherein multiple copies of the first multimeric substructure and the second multimeric substructure interact with each other at one or more non-covalent protein-protein interfaces.

20. The nanoparticle of claim 19, wherein one or more of first the polypeptides and/or one or more of the second polypeptides comprise fusion proteins, wherein the fusion proteins comprise a second functional polypeptide, including but not limited to an immunogenic portion of a polypeptide antigen, wherein the polypeptide antigen includes but is not limited to the polypeptide of claim 10 or 11.

21. The nanoparticle of claim 17, wherein the nanoparticle comprises
(a) a plurality of first polypeptides according to claim 8 or 9 that self-interact to form a first multimeric substructure; and
(b) a plurality of second polypeptides comprising or consisting of SEQ ID NO:47 that self-interact to form a second multimeric substructure;
wherein multiple copies of the first multimeric substructure and the second multimeric substructure interact with each other at one or more non-covalent protein-protein interfaces.

22. The nanoparticle of claim 21, wherein one or more of first the polypeptides and/or one or more of the second polypeptides comprise fusion proteins, wherein the fusion proteins comprise a second functional polypeptide, including but not limited to an immunogenic portion of a polypeptide antigen, wherein the polypeptide antigen includes but is not limited to the polypeptide of claim 10 or 11.

23. A composition comprising a plurality of nanoparticles according to any one of claims 17-22.

24. A nucleic acid encoding the polypeptide or fusion protein of any one of claims 1-16.

25. An expression vector comprising the nucleic acid of claim 24 operatively linked to a suitable control sequence.

26. A host cell comprising the polypeptide, fusion protein, nanoparticle, composition, nucleic acid, and/or expression vector of any one of claims 1-25.

27. A pharmaceutical composition comprising
(a) the polypeptide, fusion protein, nanoparticle, composition, nucleic acid, expression vector, and/or host cell of any one of claims 1-26; and
(b) a pharmaceutically acceptable carrier.

28. A synthetic (“degreased”) nanoparticle, comprising a cryptic transmembrane domain, wherein one or more of the hydrophobic amino acids of the cryptic transmembrane domain have been substituted with a polar amino acid.

29. The synthetic nanoparticle of claim 28, wherein the amino acid substitution is within a 19-residue sliding window for transmembrane insertion potential (dG_ins); windows of dG_ins less than or equal to +2.7 kcal/mol are confirmed to be local minima within +/−9 residues, and the cutoff of +2.7 kcal/mol is the signature of the cryptic transmembrane domain.

30. A synthetic nanoparticle, comprising a polypeptide comprising the amino acid sequence of SEQ ID NO:13.

31. The synthetic nanoparticle of any one of claims 28-30, wherein the synthetic nanoparticle is a polypeptide.

32. The synthetic nanoparticle of any one of claims 28-31, wherein the synthetic nanoparticle comprises a signal peptide.

33. The synthetic nanoparticle of any one of claims 28-32, wherein the synthetic nanoparticle comprises a tag.

34. The synthetic nanoparticle of any one of claims 28-29 and 31-33, wherein the synthetic nanoparticle comprises a one-component or homomeric nanoparticle.

35. The synthetic nanoparticle of claim 34, wherein the synthetic nanoparticle comprises an expressed sequence as shown and described herein.

36. The synthetic nanoparticle of claim 34, wherein the synthetic nanoparticle comprises variant I3-01 amino acid sequences.

37. The synthetic nanoparticle of claim 36, wherein the synthetic nanoparticle comprises a polar amino acid substitution at position 25, position, 35, position 171, position 177, or position 180, or at any two or more combinations of those positions.

38. The synthetic nanoparticle of any one of claims 28-37, wherein the synthetic nanoparticle further comprises an agent to be secreted (“secreted agent”).

39. The synthetic nanoparticle of claim 38, wherein the secreted agent is selected from:
a) a polypeptide;
b) a payload;
c) antigen displayed on the exterior of the synthetic nanoparticle

40. The synthetic nanoparticle of claim 39, wherein the polypeptide comprises an antigen or an immunogenic portion of an antigen.

41. The synthetic nanoparticle of claim 40, wherein the antigen or immunogenic portion of an antigen is of viral origin.

42. The synthetic nanoparticle of claim 41, wherein the virus is human metapneumo virus (hMPV).

43. The synthetic nanoparticle of any of claim 28-29 or 31-42, wherein the synthetic nanoparticle comprises a two-component nanoparticle.

44. The synthetic nanoparticle of claim 43, wherein the synthetic nanoparticle comprises a trimer, a tetramer, or a pentamer.

45. The synthetic nanoparticle of claim 43, wherein the synthetic nanoparticle is selected from: I53_dn5, O43-38, and I53-50.

46. The synthetic nanoparticle of claim 43, wherein the synthetic nanoparticle is I53_dn5 and wherein the pentameric subunit I53_dn5A of the synthetic nanoparticle comprises a polar amino acid substitution at at least one of position 16, position 29, position 116, position 118, or position 119, or at any two or more combinations of those positions.

47. The synthetic nanoparticle of claim 43, wherein the synthetic nanoparticle is O43-38 and wherein the tetrameric subunit O43-38tet of the synthetic nanoparticle comprises a polar amino acid substitution at position 29, position 141, position 19, position 21, or position 31, or at any two or more combinations of those positions.

48. A nucleic acid molecule encoding the synthetic nanoparticle of any previous claim.

49. The nucleic acid molecule of claim 48, wherein the polynucleotide is an mRNA.

50. An expression vector comprising the nucleic acid molecule of claim 48 or 49.

51. A cell comprising the nucleic acid molecule of claim 48 or 49 and/or the expression vector of claim 50.

52. A method of delivering a secreted agent from a cell, comprising administering or admixing the cell with the nucleic acid molecule and/or the expression vector of any preceding claim and secreting the nanoparticle or synthetic nanoparticle.

53. A vaccine comprising the nanoparticle, composition, pharmaceutical composition, synthetic nanoparticle, nucleic acid, expression vector, and/or cell of any claim herein.

54. A method to vaccinate a subject against a virus, the method comprising administering the nanoparticle, composition, pharmaceutical composition, synthetic nanoparticle(s) or the vaccine(s) described herein to the subject.

55. The method of claim 54, comprising:
(a) obtaining the nanoparticle, composition, pharmaceutical composition, synthetic nanoparticles, the compositions, or the vaccines described herein; and,
(b) administering the synthetic nanoparticles, the compositions, or the vaccines described herein to the subject.

56. The method of claim 54-55, wherein the administration elicits an immune response in the subject, such that the subject is protected against infection.

57. A kit comprising one or more components selected from the group consisting of the polypeptide, fusion protein, nanoparticle, composition, synthetic nanoparticle(s), the nucleic acid molecule(s), the expression vector(s), the cell(s), the composition(s), or the vaccine(s) described herein.

58. A computer-implemented method for designing a secreted peptide, comprising:
generating a 3D structure of a protein of interest with a 19-residue sliding window for transmembrane insertion potential (dG_ins);
Windows of dG_ins less than or equal to +2.7 kcal/mol are confirmed to be local minima within +/−9 residues, and the cutoff of +2.7 kcal/mol is the signature of a cryptic transmembrane domain;
designing one or more peptide sequences based on the generated 3D structure and predicting mutations at each position within that domain, wherein allowed residues are all polar, excluding histidine, such that the final allowable residues are amino acids D, E, K, R, Q, N, S, T, Y; and side chains of other residues within an 8-Angstrom shell are allowed to adopt different rotamers (“repack” to one of skill in the art) but not mutate to other residues (“design” to one of skill in the art).

59. The computer-implemented method of claim 58, wherein for each mutation or set of mutations, the score of the overall energy of the structure is generated and wherein
(a) if the new score is higher than the original score by a threshold amount of 15 REU (dscore), the degreaser variant is discarded and not further evaluated; or
(b) if the new score is within the tolerance, but the change in dG_ins is less than +0.27 kcal/mol (ddG_ins), the mutation placed at that position is rejected and disallowed at that position, and the position is subjected to mutation again; or
(c) if the new score is within the tolerance and the ddG_ins is greater than +0.27 kcal/mol, the mutation is accepted, the structure is optionally output, and the metrics of that mutation are written to the final output file.


SEQUENCE LISTING (62 sequences)

SEQ ID NO: 1 - protein, 205 aa, Artificial Sequence
  Synthetic
  MISC_FEATURE (1)..(1): optionally absent
MKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 2 - protein, 214 aa, Artificial Sequence
  Synthetic
  MISC_FEATURE (1)..(1): optionally absent
MERNKLARQIIDTCLEMTRLGLNQGTAGSVSVRYQDGMLITPTGIPYEKLTESHIVFIDGNGKHEEGKLPQSEWRFHMAAYKARPDANAVVHNHAVHCTAVSILNRSIPAIHYMIAAAGGNSIPCAPAATFGTDELSMLVAVALLNRKATLLQHHGLIACEVNLEKALWLAHEVEVLAQLYLTTLAITDPVPVLSDEEIAVVLEKFKTFGLRIE

SEQ ID NO: 3 - protein, 213 aa, Artificial Sequence
  Synthetic
  MISC_FEATURE (1)..(1): optionally absent
  MISC_FEATURE (206)..(213): optionally absent
MKMEELFKKHKIVAVLRANSVVLALAKALAVFLGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGEPEEVREKAKAFVEKIRGCTELEHHHHHH

SEQ ID NO: 4 - protein, 155 aa, Artificial Sequence
  Synthetic
  MISC_FEATURE (1)..(2): optionally absent
MGKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 5 - protein, 530 aa, Artificial Sequence
  Synthetic
  MISC_FEATURE (1)..(20): optionally absent
  MISC_FEATURE (490)..(530): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILTAVLGSTMILVSVFIIIKKTKKPTGAPPELSGV

SEQ ID NO: 6 - protein, 542 aa, Artificial Sequence
  Synthetic
  MISC_FEATURE (1)..(20): optionally absent
  MISC_FEATURE (490)..(542): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGGGGSGYIPEAPRDGQAYVRKDGEWVLLSTFLGGIEGRHHHHHH

SEQ ID NO: 7 - protein, 244 aa, Artificial Sequence
  Synthetic
  MISC_FEATURE (1)..(26): optionally absent
  MISC_FEATURE (230)..(244): optionally absent
METDTLLLWVLLLWVPGSTGDYKDEKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLHHHHHH

SEQ ID NO: 8 - protein, 244 aa, Artificial Sequence
  Synthetic
  MISC_FEATURE (1)..(26): optionally absent
  MISC_FEATURE (230)..(244): optionally absent
METDTLLLWVLLLWVPGSTGDYKDEKMEELFKKHKIVAVLRANSVEEAKKDALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLHHHHHH

SEQ ID NO: 9 - protein, 244 aa, Artificial Sequence
  Synthetic
  MISC_FEATURE (1)..(26): optionally absent
  MISC_FEATURE (230)..(244): optionally absent
METDTLLLWVLLLWVPGSTGDYKDEKMEELFKKHKIVAVLRANSVEEAKKNALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLHHHHHH

SEQ ID NO: 10 - protein, 244 aa, Artificial Sequence
  Synthetic
  MISC_FEATURE (1)..(26): optionally absent
  MISC_FEATURE (230)..(244): optionally absent
METDTLLLWVLLLWVPGSTGDYKDEKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGSALVKGTPVEVAEKAKAFVEKIRGCTEQKLISEEDLHHHHHH

SEQ ID NO: 11 - protein, 244 aa, Artificial Sequence
  Synthetic
  MISC_FEATURE (1)..(26): optionally absent
  MISC_FEATURE (230)..(244): optionally absent
METDTLLLWVLLLWVPGSTGDYKDEKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTEQKLISEEDLHHHHHH

SEQ ID NO: 12 - protein, 244 aa, Artificial Sequence
  Synthetic
  MISC_FEATURE (1)..(26): optionally absent
  MISC_FEATURE (230)..(244): optionally absent
METDTLLLWVLLLWVPGSTGDYKDEKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTEQKLISEEDLHHHHHH

SEQ ID NO: 13 - protein, 239 aa, Artificial Sequence
  Synthetic
  MISC_FEATURE (1)..(21): optionally absent
  MISC_FEATURE (225)..(239): optionally absent
METDTLLLWVLLLWVPGSTGDMEELFKEHKIVAVLRANSVEEAKKKALAVFLGGVHLIEITFTVPDADTVIKELSFLKEMGAIIGAGTVTSVEQAREAVESGAEFIVSPHLDEEISQFAKEEGVFYMPGVMTPTELVKAMKLGHTILKLFPGEVVGPQFVEAMKGPFPNVKFVPTGGVNLCNVAEWFEAGVLAVGVGSALVEGTPVEVAEKAKAFVEKIEGATEQKLISEEDLHHHHHH

SEQ ID NO: 14 - protein, 239 aa, Artificial Sequence
  Synthetic
  MISC_FEATURE (1)..(21): optionally absent
  MISC_FEATURE (225)..(239): optionally absent
METDTLLLWVLLLWVPGSTGDMEELFKEHKIVAVLRANSVEEAKKKALAVFLGGVDLIEITFTVPDADTVIKELSFLKEMGAIIGAGTVTSVEQAREAVESGAEFIVSPHLDEEISQFAKEEGVFYMPGVMTPTELVKAMKLGHTILKLFPGEVVGPQFVEAMKGPFPNVKFVPTGGVNLDNVAEWFEAGVQAVGVGEALNEGTPVEVAEKAKAFVEKIEGATEQKLISEEDLHHHHHH

SEQ ID NO: 15 - protein, 199 aa, Artificial Sequence
  Synthetic
  MISC_FEATURE (1)..(25): optionally absent
  MISC_FEATURE (180)..(199): optionally absent
METDTLLLWVLLLWVPGSTGDYKDEKKYDGSKLRIGILHARENAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFNLEGSEQKLISEEDLHHHHHH

SEQ ID NO: 16 - protein, 199 aa, Artificial Sequence
  Synthetic
  MISC_FEATURE (1)..(25): optionally absent
  MISC_FEATURE (180)..(199): optionally absent
METDTLLLWVLLLWVPGSTGDYKDEKKYDGSKLRIGILHARWNAEIILALVLGANKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFNLEGSEQKLISEEDLHHHHHH

SEQ ID NO: 17 - protein, 199 aa, Artificial Sequence
  Synthetic
  MISC_FEATURE (1)..(25): optionally absent
  MISC_FEATURE (180)..(199): optionally absent
METDTLLLWVLLLWVPGSTGDYKDEKKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLNCDKDEQAEARAGLIEGKMHNHGEDWGAAAVEMATKFNLEGSEQKLISEEDLHHHHHH

SEQ ID NO: 18 - protein, 199 aa, Artificial Sequence
  Synthetic
  MISC_FEATURE (1)..(25): optionally absent
  MISC_FEATURE (180)..(199): optionally absent
METDTLLLWVLLLWVPGSTGDYKDEKKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLNCLTDEQAEARAGLIEGKMHNHGEDWGAAAVEMATKFNLEGSEQKLISEEDLHHHHHH

SEQ ID NO: 19 - protein, 199 aa, Artificial Sequence
  Synthetic
  MISC_FEATURE (1)..(25): optionally absent
  MISC_FEATURE (180)..(199): optionally absent
METDTLLLWVLLLWVPGSTGDYKDEKKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCDTDEQAEARAGLIEGKMHNHGEDWGAAAVEMATKFNLEGSEQKLISEEDLHHHHHH

SEQ ID NO: 20 - protein, 199 aa, Artificial Sequence
  Synthetic
  MISC_FEATURE (1)..(25): optionally absent
  MISC_FEATURE (180)..(199): optionally absent
METDTLLLWVLLLWVPGSTGDYKDEKKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLKDEQAEARAGLIEGKMHNHGEDWGAAAVEMATKFNLEGSEQKLISEEDLHHHHHH

SEQ ID NO: 21 - protein, 200 aa, Artificial Sequence
  Synthetic
  MISC_FEATURE (1)..(27): optionally absent
  MISC_FEATURE (181)..(200): optionally absent
METDTLLLWVLLLWVPGSTGDYKDEMGKYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFNLEGSEQKLISEEDLHHHHHH

SEQ ID NO: 22 - protein, 200 aa, Artificial Sequence
  Synthetic
  MISC_FEATURE (1)..(27): optionally absent
  MISC_FEATURE (181)..(200): optionally absent
METDTLLLWVLLLWVPGSTGDYKDEMGKYDGSKLRIGILHARENAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFNLEGSEQKLISEEDLHHHHHH

SEQ ID NO: 23 - protein, 197 aa, Artificial Sequence
  Synthetic
  MISC_FEATURE (1)..(27): optionally absent
  MISC_FEATURE (178)..(197): optionally absent
METDTLLLWVLLLWVPGSTGDYKDEMGKYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFNLEGSEQKLISEEDLHHHHHH

SEQ ID NO: 24 - protein, 259 aa, Artificial Sequence
  Synthetic
  MISC_FEATURE (1)..(28): optionally absent
  MISC_FEATURE (243)..(259): optionally absent
METDTLLLWVLLLWVPGSTGDYKDESGMERNKLARQIIDTCLEMTRLGLNQGTAGSVSVRYQDGMLITPTGIPYEKLTESHIVFIDGNGKHEEGKLPQSEWRFHMAAYKARPDANAVVHNHAVHCTAVSILNRSIPAIHYMIAAAGGNSIPCAPAATFGTDELSMLVEVALLNRKATLLQHHGLIACEVNLEKALWLAHEVEVLAQLYLTTLAITDPVPVLSDEEIAVVLEKFKTFGLRIEEGSQKLISEEDLHHHHHH

SEQ ID NO: 25 - protein, 265 aa, Artificial Sequence
  Synthetic
  MISC_FEATURE (1)..(28): optionally absent
  MISC_FEATURE (243)..(265): optionally absent
METDTLLLWVLLLWVPGSTGDYKDESGMNRNELARQIIDTMKEMTRLGLNQGTAGSVSVRYQDGMLITPIGIPYEKLTEDHIVFIDGNGKHEEGKLPQSEWRFHMAAYKARPDANAVVHNHAVHSTAVSILNREIPAIHYMIAAAGGNSIPSAPAATFGTDELSMLVEVALLDRKATLLQHHGLIAVETNLEKALWLAHEVEVLAQLYLTTLAITDPVPVLSDEEIKTVLEKFKTFGLRIEEGSQKLISEEDLGSGSGSHHHHHH

SEQ ID NO: 26 - protein, 245 aa, Artificial Sequence
  Synthetic
  MISC_FEATURE (1)..(26): optionally absent
  MISC_FEATURE (228)..(245): optionally absent
METDTLLLWVLLLWVPGSTGDYKDEKMEELFKKHKIVAVLRANSDVLALAKALAVFLGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGEPEEVREKAKAFVEKIRGCGSEQKLISEEDLHHHHHH

SEQ ID NO: 27 - protein, 245 aa, Artificial Sequence
  Synthetic
  MISC_FEATURE (1)..(26): optionally absent
  MISC_FEATURE (228)..(245): optionally absent
METDTLLLWVLLLWVPGSTGDYKDEKMEELFKKHKIVAVLRANSVVEALAKALAVFLGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGEPEEVREKAKAFVEKIRGCGSEQKLISEEDLHHHHHH

SEQ ID NO: 28 - protein, 245 aa, Artificial Sequence
  Synthetic
  MISC_FEATURE (1)..(26): optionally absent
  MISC_FEATURE (228)..(245): optionally absent
METDTLLLWVLLLWVPGSTGDYKDEKMEELFKKHKIVAVLRANSVVLALAKALAVFNGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGEPEEVREKAKAFVEKIRGCGSEQKLISEEDLHHHHHH

SEQ ID NO: 29 - protein, 489 aa, Artificial Sequence
  Synthetic
  MISC_FEATURE (1)..(18): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNT

SEQ ID NO: 30 - protein, 768 aa, Artificial Sequence
  Synthetic
  MISC_FEATURE (1)..(18): optionally absent
  MISC_FEATURE (490)..(556): optionally absent
  MISC_FEATURE (761)..(768): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGGGGSGYIPEAPRDGQAYVRKDGEWVLLSTFLGSGGSGSGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTELEHHHHHH

SEQ ID NO: 31 - protein, 489 aa, Artificial Sequence
  Synthetic
  MISC_FEATURE (1)..(18): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNT

SEQ ID NO: 32 - protein, 768 aa, Artificial Sequence
  Synthetic
  MISC_FEATURE (1)..(18): optionally absent
  MISC_FEATURE (490)..(556): optionally absent
  MISC_FEATURE (761)..(768): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGGGGSGYIPEAPRDGQAYVRKDGEWVLLSTFLGSGGSGSGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTELEHHHHHH

SEQ ID NO: 33 - protein, 489 aa, Artificial Sequence
  Synthetic
  MISC_FEATURE (1)..(18): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNT

SEQ ID NO: 34 - protein, 768 aa, Artificial Sequence
  Synthetic
  MISC_FEATURE (1)..(18): optionally absent
  MISC_FEATURE (490)..(556): optionally absent
  MISC_FEATURE (761)..(768): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGGGGSGYIPEAPRDGQAYVRKDGEWVLLSTFLGSGGSGSGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTELEHHHHHH

SEQ ID NO: 35 - protein, 489 aa, Artificial Sequence; identical to SEQ ID NO: 31
  Synthetic
  MISC_FEATURE (1)..(18): optionally absent

SEQ ID NO: 36 - protein, 632 aa, Artificial Sequence
  Synthetic
  MISC_FEATURE (1)..(18): optionally absent
  MISC_FEATURE (490)..(501): optionally absent
  MISC_FEATURE (621)..(632): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 37 - protein, 489 aa, Artificial Sequence; identical to SEQ ID NO: 31
  Synthetic
  MISC_FEATURE (1)..(18): optionally absent

SEQ ID NO: 38 - protein, 633 aa, Artificial Sequence
  Synthetic
  MISC_FEATURE (1)..(18): optionally absent
  MISC_FEATURE (490)..(502): optionally absent
  MISC_FEATURE (622)..(633): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGSEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 39 - protein, 489 aa, Artificial Sequence; identical to SEQ ID NO: 31
  Synthetic
  MISC_FEATURE (1)..(18): optionally absent

SEQ ID NO: 40 - protein, 634 aa, Artificial Sequence
  Synthetic
  MISC_FEATURE (1)..(18): optionally absent
  MISC_FEATURE (490)..(503): optionally absent
  MISC_FEATURE (623)..(634): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGSGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 41 - protein, 490 aa, Artificial Sequence
  Synthetic
  MISC_FEATURE (1)..(18): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGDSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNT

SEQ ID NO: 42 - protein, 633 aa, Artificial Sequence
  Synthetic
  MISC_FEATURE (1)..(18): optionally absent
  MISC_FEATURE (491)..(502): optionally absent
  MISC_FEATURE (622)..(633): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGDSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 43 - protein, 490 aa, Artificial Sequence; identical to SEQ ID NO: 41
  Synthetic
  MISC_FEATURE (1)..(18): optionally absent

SEQ ID NO: 44 - protein, 634 aa, Artificial Sequence
  Synthetic
  MISC_FEATURE (1)..(18): optionally absent
  MISC_FEATURE (491)..(503): optionally absent
  MISC_FEATURE (623)..(634): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGDSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGSEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 45 - protein, 490 aa, Artificial Sequence; identical to SEQ ID NO: 41
  Synthetic
  MISC_FEATURE (1)..(18): optionally absent

SEQ ID NO: 46 - protein, 635 aa, Artificial Sequence
  Synthetic
  MISC_FEATURE (1)..(18): optionally absent
  MISC_FEATURE (491)..(504): optionally absent
  MISC_FEATURE (624)..(635): optionally absent
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGDIALGRAEAAARTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGDSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGSGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 47 - protein, 120 aa, Artificial Sequence
  Synthetic
  MISC_FEATURE (1)..(1): optionally absent
MEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 48 - protein, 21 aa, Artificial Sequence
  Synthetic
METDTLLLWVLLLWVPGSTGD

SEQ ID NO: 49 - protein, 26 aa, Artificial Sequence
  Synthetic
METDTLLLWVLLLWVPGSTGDYKDEK

SEQ ID NO: 50 - protein, 6 aa, Artificial Sequence
  Synthetic
HHHHHH

SEQ ID NO: 51 - protein, 10 aa, Artificial Sequence
  Synthetic
EQKLISEEDL

SEQ ID NO: 52 - protein, 19 aa, Artificial Sequence
  Synthetic
KLNFELGIPVIFGVLNCDK

SEQ ID NO: 53 - protein, 19 aa, Artificial Sequence
  Synthetic
KLNFELGIPVIFGVLNCLT

SEQ ID NO: 54 - protein, 19 aa, Artificial Sequence
  Synthetic
KLNFELGIPVIFGVLTCDT

SEQ ID NO: 55 - protein, 19 aa, Artificial Sequence
  Synthetic
KLNFELGIPVIFGVLTCLK

SEQ ID NO: 56 - protein, 19 aa, Artificial Sequence
  Synthetic
KLNFELGIPVIFGVLTCLT

SEQ ID NO: 57 - protein, 19 aa, Artificial Sequence
  Synthetic
ARWNAEIILALVEGALKRL

SEQ ID NO: 58 - protein, 19 aa, Artificial Sequence
  Synthetic
ARENAEIILALVLGALKRL

SEQ ID NO: 59 - protein, 19 aa, Artificial Sequence
  Synthetic
ARWNAEIILALVLGANKRL

SEQ ID NO: 60 - protein, 19 aa, Artificial Sequence
  Synthetic
ARWNAEIILALVLGALERL

SEQ ID NO: 61 - protein, 19 aa, Artificial Sequence
  Synthetic
ARWNAEIILALVLGALKRL

SEQ ID NO: 62 - protein, 9 aa, Artificial Sequence
  Synthetic
DEKRQNSTY